(19) United States
(12) Patent Application Publication
(10) Pub. No.: US 2022/0403043 A1
(43) Pub. Date: Dec. 22, 2022

(54) MULTISPECIFIC ANTIBODIES FOR USE IN TREATING DISEASES

(71) Applicant: Yeda Research and Development Co. Ltd., Rehovot (IL)

(72) Inventors: Rony DAHAN, Rehovot (IL); Ran SALOMON, Rehovot (IL)

(73) Assignee: Yeda Research and Development Co. Ltd., Rehovot (IL)

(21) Appl. No.: 17/870,876

(22) Filed: Jul. 22, 2022

Related U.S. Application Data
(63) Continuation of application No. PCT/IL2021/050064, filed on Jan. 21, 2021.

(30) Foreign Application Priority Data
Jan. 22, 2020 (IL) 272194

(51) Int. Cl. C07K 16/28; A61P 35/00

(52) U.S. Cl. CPC C07K 16/2896; A61P 35/00; A61K 2039/505

(57) ABSTRACT

A multispecific antibody is provided. The antibody comprising a first moiety, which binds and activates CD40, a second moiety, which specifically binds a dendritic cell (DC) and a third moiety comprising a modified Fc region of the multispecific antibody for enhancing specificity and affinity of binding to FcγRIIb and uses of same.

### RELATED APPLICATIONS

This application is a Continuation of PCT Patent Application No. PCT/IL2021/050064, having international filing date of Jan. 21, 2021 which claims the benefit of priority of Israeli Patent Application No. 272194 filed on Jan. 22, 2020. The contents of the above applications are all incorporated by reference as if fully set forth herein in their entirety.

### SEQUENCE LISTING STATEMENT

The XML file, entitled 93177Sequence Listing.xml, created on Jul. 22, 2022, comprising 76,136 bytes, submitted concurrently with the filing of this application is incorporated herein by reference.

### FIELD AND BACKGROUND OF THE INVENTION

The present invention, in some embodiments thereof, relates to multispecific antibodies for use in treating diseases.

Activation of the immune system in order to eradicate tumor cells can be achieved by either blocking inhibitory checkpoint molecules, like PD-1, or activating stimulatory molecules, like CD40 [1][2]. When crosslinked by its natural ligand CD40L, CD40 a member of the tumor necrosis factor receptor (TNFR) family stimulates immune responses including the generation of cytotoxic T cells [3][4]. Agonistic anti-CD40 Abs mimicking CD40L have been proposed as an efficient approach to crosslink CD40 and thereby promote the maturation of DCs and the subsequent generation of tumor antigen specific cytotoxic T cells. Mechanistically, CD40 activation is a proximal event in T cell priming and, thus, anti-CD40 Abs may be critical in converting cold tumors to hot and generating effective T cell immunity [3]. Indeed, antitumor activities of agonistic anti-CD40 Abs have been demonstrated to be effective in many animal models of different tumors [5].

A number of previous publications demonstrated that engagement of the inhibitory FcγRIIB is an absolute requirement for the in-vivo antitumor activity of agonistic Abs targeting mouse CD40, as well as other members of the TNFR family. This is due to the high-order crosslinking of the CD40 antibody by FcγRIIB expressed on neighboring cells. Such crosslinking enhances the clustering of CD40 on the cell surface and results in enhanced CD40 signaling [6] [7]. However, a similar requirement for human Abs has been questioned by several recent studies. The limitation of these studies is that in vitro experimental systems were used. Alternatively, these human Abs were evaluated in wild type mice, thereby limiting their ability to mimic the complex and unique cellular distribution, binding affinities, and functionality of human FcγRs.

CP-870,893 is a human anti-CD40 Ab under clinical evaluation which composed of the anti-CD40 clone 2141 on a human IgG2 isotype. is a potent agonistic anti-human CD40 Ab but yet surprisingly displayed only modest antitumor activities in patients with pancreatic ductal adenocarcinoma (PDA) or other solid tumors [8] [9]. It was found that the modest in vivo activity of CP-870,893 can be attributed to the fact that its Fc domain is of the human IgG2 subtype and only weakly interacts with human FcγRIIB [10]. Of particular significance is the observation that the antitumor activities of agonistic anti-CD40 Abs can be enhanced through FcγRIIB-targeted Fc engineering. In previous works, a number of Fc engineered variants of 2141 were tested on an isogenic mouse model fully humanized for CD40 and FcγRs on a background lacking the mouse homologues of these receptors and selected the “V11” Fc variant, that selectively enhanced FcγRIIB binding, as the optimal clinical candidate. The Fc-engineered V11 version of 2141 had significantly enhanced immunostimulatory activity in vivo that translated to superior anti-tumor potency in several types of murine tumor models compared to the original IgG2 subclass of 2141 [10]. When used in patients at their respective maximum tolerated doses (MTDs), increased therapeutic antitumor immunity can be achieved by the Fc-engineered 2141-V11 compared to its parental IgG2 version, however an optimal dose may never be reached for either subclass in humans when used at their respective MTDs. In preparation for clinical studies of Fc-engineered version of 2141 antibody the dosing and delivery regimen were optimized to result in minimal toxicity with optimal antitumor activity. Intaratumoral administration of the Fc-engineered 2141 resulted with such maximal therapeutic window compared to that of the parental 2141 Ab and of systemic Ab administration [11]. Based on these studies a 2nd-generation Fc-engineered version of 2141 (Fc-engineered “F11”) was developed and administered by intratumoral injections in patients with solid tumors (ClinicalTrials(dot)gov Identifier: NCT04059588). While promising for some patients, intratumoral administration is not suitable to all patients, and may be limited to patients with locally or metastatic solid tumors to the skin and tumor that accessible to radiographically directed therapy.

Additional Background Art:
U.S. 20160376371;
U.S. 20170253659;
WO2017004016;
WO2018213747;
Mazor, Yariv, et al. “Improving target cell specificity using a novel monovalent bispecific IgG design.” MAbs. Vol. 7. No. 2. Taylor & Francis, 2015.

### SUMMARY OF THE INVENTION

According to an aspect of some embodiments of the present invention there is provided a multispecific antibody comprising a first moiety, which binds and activates CD40 and a second moiety, which specifically binds a dendritic cell (DC).

According to some embodiments of the invention, the multispecific antibody is a bispecific antibody.

According to some embodiments of the invention, the second moiety binds a DC marker selected from the group consisting of CD11c, CD11b, DEC-205, BDCA-1, CD8, CD8α, CD103 and MHC-ClassII (e.g., HLA-DR), CD141, FLT3, CD13, CD1c, Clec9a, and XCR1.

According to some embodiments of the invention, the second moiety binds CD11c.

According to some embodiments of the invention, the second moiety binds DEC-205.
According to some embodiments of the invention, the second moiety binds Clec9a.

According to some embodiments of the invention, the second moiety binds XCR1.

According to some embodiments of the invention, the multispecific antibody comprises a first moiety comprising complementary determining regions as set forth in SEQ ID NOs: 19-21 in a heavy chain with an N to C orientation and complementary determining regions as set forth in SEQ ID NOs: 22-24 in a light chain with an N to C orientation.

According to some embodiments of the invention, the multispecific antibody is a trifunctional antibody.

According to some embodiments of the invention, the multispecific antibody comprises a third moiety comprising a modified Fc region of the multispecific antibody for enhancing specificity and affinity of binding to FcγRIIb.

According to some embodiments of the invention, the modified Fc region comprises mutations as in SEQ ID NO: 2.

According to some embodiments of the invention, the multispecific antibody comprises knobs-into-holes mutations.

According to some embodiments of the invention, the mutations are in a CH3 domain of a first antibody of the bispecific antibody comprising Y349C/T366S/L368A/Y407V and in a CH3 domain of a second antibody of the multispecific antibody comprising S354C/T366W.

According to some embodiments of the invention, the multispecific antibody comprises SEQ ID NOs: 5 and 6 and either of SEQ ID NOs: 37 and 38; SEQ ID NOs: 39 and 40; SEQ ID NOs: 15 and 16; or SEQ ID NOs: 17 and 18.

According to an aspect of some embodiments of the present invention there is provided a pharmaceutical composition comprising the multispecific antibody.

According to an aspect of some embodiments of the present invention there is provided a nucleic acid sequence encoding a heavy and/or light chain of the multispecific antibody.

According to an aspect of some embodiments of the present invention there is provided an expression vector comprising the nucleic acid sequence.

According to an aspect of some embodiments of the present invention there is provided a cell transformed with the expression vector.

According to an aspect of some embodiments of the present invention there is provided a method of preparing a multispecific antibody comprising:
(a) culturing the cell under conditions which allow the expression of the multispecific antibody; and
(b) isolating the multispecific antibody from the cell.

According to an aspect of some embodiments of the present invention there is provided a method of stimulating an immune response in a subject in need thereof, the method comprising administering to the subject a therapeutically effective amount of the pharmaceutical composition, thereby stimulating the immune response in the subject.

According to some embodiments of the invention, the subject has a tumor and an immune response against the tumor is stimulated.

According to some embodiments of the invention, the subject has a chronic viral infection and an immune response against the viral infection is stimulated.

According to an aspect of some embodiments of the present invention there is provided a method of treating cancer in a subject in need thereof, the method comprising administering to the subject a therapeutically effective amount of the pharmaceutical composition, thereby treating cancer in the subject.

According to some embodiments of the invention, the cancer is selected from the group consisting of: bladder cancer, breast cancer, uterine/cervical cancer, ovarian cancer, prostate cancer, testicular cancer, esophageal cancer, gastrointestinal cancer, pancreatic cancer, colorectal cancer, colon cancer, kidney cancer, head and neck cancer, lung cancer, stomach cancer, germ cell cancer, bone cancer, liver cancer, thyroid cancer, skin cancer, neoplasm of the central nervous system, lymphoma, leukemia, myeloma, sarcoma, and virus-related cancer.

According to an aspect of some embodiments of the present invention there is provided a method of treating a chronic viral infection in a subject in need thereof, the method comprising administering to the subject a therapeutically effective amount of the pharmaceutical composition, thereby treating the chronic viral infection in the subject.

According to an aspect of some embodiments of the present invention there is provided the pharmaceutical composition for use in the treatment of cancer and/or a chronic viral infection.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1A shows the variable domain sequences of antibodies (Abs) used according to some embodiments of the invention in a multispecific antibody configuration. The sequence of variable heavy chain (VH) and variable light chain (VL) domains of N418 and HD-109 Abs were sequenced from the respective hybridoma's RNA by amplification of cDNA ends method (“anchored” PCR). The sequence of 2141 was previously identified from the patent application of this Ab. The sequences of CDRs are underlined. The sequence listing identification is SEQ ID NOs: 78 and 38 for 2141, SEQ ID NOs: 7 and 8 for N418, SEQ ID NOs: 39 and 40 for HD109, SEQ ID NOs: 15 and 16 for 10B4.

FIG. 1B shows sequences of antibodies or fragments thereof which can be used according to some embodiments of the invention.

FIG. 2 shows the constructs generated for production of monospecific and bispecific Abs. Variable domains were amplified from hybridoma's cDNA (N418 and HD-109) or de-novo synthesized (2141) and cloned in-frame with IgG1 constant domains inside expression vectors, as illustrated. VH, Variable domain Heavy chain; VL, Variable domain Light chain; CH1-3, Constant domain Heavy chains 1-3; CL, Constant domain Light chain; KiH, knobs-into-holes mutations.

FIGS. 3A-B show SDS-PAGE and Analytical size-exclusion chromatograms of monospecific and bispecific Abs. (FIG. 3A) SDS-PAGE analysis of the five Abs. The homogenous band of the non-reduced sample confirms the assembly of N418 and HD-109 with 2141 Abs into one heterodimer. Reduction of the sample confirm that the bispecific heterodimer composes of the four Ab chains, two from each partner Ab. (FIG. 3B) Analytical size-exclusion chromatograms of monospecific and bispecific Abs, numbers indicate molecular weight of the Abs.

FIGS. 4A-D show dual antigen binding properties of the bispecific Abs. (FIG. 4A) ELISA binding to hCD40. Standard binding ELISA titration assay of the anti-CD40 (red) monospecific and anti-CD40/CD11c (black)-CD40/DEC-205 (blue) bispecific Abs to recombinant huCD40 protein. The anti-CD40/CD11c-CD40/DEC-205 bispecific Abs recognize huCD40 similar to the parental monospecific anti-CD40 Ab. (FIG. 4B) ELISA binding to DEC-205 or CD11c. Standard binding ELISA titration assay of the anti-CD40 (red)—DEC-205 or CD11c (black) monospecific and anti-CD40/DEC-205 or CD40/CD11c (blue) bispecific Abs to recombinant DEC-205 or CD11c proteins. Both anti-CD40/DEC-205 or CD40/CD11c bispecific Ab and the parental monospecific anti-DEC-205 or CD11c Ab but not the monospecific anti-CD40 Ab, recognize DEC-205 or CD11c respectively. (FIG. 4C) Simultaneous ELISA binding to DEC-205 or CD11c and huCD40. Simultaneous binding sandwich ELISA titration assay of the anti-CD40 (red)-DEC-205 or CD11c (black) monospecific and anti-CD40/DEC-205 or CD40/CD11c (blue) bispecific Abs to recombinant DEC-205 or CD11c and huCD40 proteins. Only the anti-CD40/DEC-205 and CD40/CD11c bispecific Abs binds simultaneously to both proteins respectively. (D) CD40/CD11c (2141/N418) bsAb and 2141 were used to stain splenocytes from humanized CD40/FcgR mice. The bsAb has preferred binding to DCs and reduced binding to B cells compared to the parental CD40 mAb, as measured by CD19 gating.

FIG. 5 shows in vitro stimulation of DCs with anti human CD40 bispecific Abs. Human DCs were activated by the CD40/DEC-205 bsAb. Activation was detected by upregulation of activation of immature human DCs cultured with the indicated bsAbs. Activation was determined by upregulation of different surface activation markers (CD86 and CD54 are shown). Representative data from four donors.

FIGS. 6A-B show that FcγR-mediated crosslinking is required for CD40/DCs bsAb activity. (A) Immature human DCs were incubated with increased doses of the indicated Fc variants of anti-human CD40/DEC-205 or CD40/CD11c bsAbs. Upregulation of CD86 and CD54 activation markers was analyzed by flow cytometry. Representative data from one out of four donors is shown. (B) T cell activation determined by flow cytometry analysis for OVA-specific CD8⁺ T cells in the blood of hCD40/FcγR mice immunized with OVA in the presence of the indicated Fc variants of CD40/DCs bsAbs. Each dot represents an individual mouse. Data are displayed as the mean±SEM. *p≤0.05, **p≤0.01.

FIGS. 7A-D show improved therapeutic window by reducing liver toxicity and increasing activity using bsAbs. (FIG. 7A) Dose dependent T cell activation assay determined by flow cytometry analysis for OVA-specific CD8+ T cells in the blood of humanized CD40/FcgR mice immunized with OVA in the presence of the indicated anti-CD40 mAb or bsAbs. Each dot represents an individual mouse. (FIG. 7B) Does dependent toxicity of liver transaminases in response to increasing levels of anti-CD40 antibodies. Mice were treated with increasing doses of anti-CD40 mAb or bsAbs and liver transaminases (AST and ALT) were measured. Each dot represents an individual mouse. (FIG. 7C) CD40/DC bsAbs have an improved liver toxicity profile compared to that of the monospecific 2141 CD40 Ab. Efficacy axis represent the mean of OVA-specific CD8+ T cells in the blood of humanized CD40/FcgR mice indicated in panel A. Liver toxicity axis represent the mean of AST, ALT liver transaminases indicated in panel B. (FIG. 7D) Determination of the MTD for liver toxicity in humanized mice allows significantly improved T cells activity without over toxicity of CD40/CD11c bsAb compared to the monospecific CD40 mAb. T cell activation assay determined by flow cytometry analysis for OVA-specific CD8+ T cells in the blood of humanized CD40/FcgR mice immunized with OVA in the presence of the indicated anti-CD40 mAb or bsAbs. (Upper panel). Liver transaminases in response to anti-CD40 antibodies. Mice were treated with anti-CD40 mAb or bsAbs and liver transaminases (AST and ALT) were measured. Each dot represents an individual mouse. (Lower panel).

FIGS. 8A-H show cell populations that mediate efficacy and toxicity of CD40 mAb. (A, B) T cell activation following CD40 mAb treatment determined by flow cytometry for OVA-specific CD8⁺ blood T cells of C57BL/6 (green) and Batf3^{-/-} (blue) mice immunized with OVA (A), or inoculated with B16-OVA tumor (B). Left: Representative flow plots gated on CD8⁺ cells showing mean±SEM. Right: Percentages of gated cells; each dot represents an individual mouse. (C) C57BL/6 and Batf3^{-/-} mice inoculated with MC38 or MCA-205 tumor cells and treated with CD40 mAb. Results are presented as means±SEM (n=8-13 per group). (D) Liver transaminase blood levels following CD40 mAb injection in the indicated strains. (E+F) hCD40/FcγR mice were injected with clodronate liposomes (E), or anti-CD42b (F), 24 hours prior to CD40 mAb injection. Blood AST and ALT levels were measured (left), and livers were harvested and analyzed (right, representative liver H&E sections; scale bars=200 μm). (G) hCD40/FcγR mice were injected with clodronate liposomes 24 hours prior to CD40 mAb injection. Blood platelet count after 24 hours. (H) hCD40/FcγR mice were injected with CD40 mAb, and after 2.5 hours, the LN and spleen were harvested; single cell suspensions were analyzed by flow cytometry for intracellular IL-6 expression. Top: Representative cell sorter plots. Bottom: Grouped IL-6 staining intensities. Data represent mean±SEM. *P≤0.05, **p≤0.01, ***P≤0.001, ****P≤0.0001, ns; non-significant.

FIG. 8I shows expression of CD40 on the indicated cells in MC38 tumor-bearing mice. Tumor, draining lymph node (LN), spleen and liver were harvested for flow cytometry analysis. Kupffer cells (KCs), macrophages (MFs), Dendritic cells (DCs), conventional type 1 DC (cDC1), conventional type 2 DC (cDC2). Delta geometric mean fluorescence intensities (ΔMFIs) shown.

FIGS. 8J-K show bsAb target selection. Expression of CD11c and DEC-205 on the indicated cell types in MC38 tumor, draining lymph node (LN), spleen and liver, of tumor-bearing mice, and on platelets (K) of naïve mice. CD41 served as a positive control marker for platelets. DEC-205 geometric mean fluorescence intensities (MFIs) and CD11c delta geometric mean fluorescence intensities (ΔMFIs) are shown. Each dot represents an individual mouse (J). FACS analysis of a representative mouse is shown in (K).

FIGS. 8L-R show cell populations mediating efficacy and toxicity of CD40 mAb. (L) Quantity of Kupffer cells in the liver of C57BL/6 (green) or Batf3^{-/-} (blue) mice was analyzed by flow cytometry. (M) C57BL/6 mice were injected with clodronate liposomes. After 24 hr, livers were harvested; single cell suspensions were analyzed by flow cytometry for the frequencies of the indicated cell populations. (N) C57BL/6 mice were injected with clodronate liposomes 24 hr prior to CD40 mAb injection. After 24 hr, blood AST and ALT levels were measured. (O-P) hCD40/FcγR mice were injected with anti-CD42b 24 hr prior to CD40 mAb injection. Platelets were measured after 24 hr (O), and livers were harvested and analyzed (P). Representative liver H&E section; scale bar=100 μm. (Q) Serum IL-6 and TNF-α levels after CD40 mAb injections. hCD40/FcγR mice were injected with 2141 CD40 mAb, and serum was collected after 3 hr. Cytokine levels were determined by ELISA. (R) Intracellular IL-6 expression after CD40 mAb injections. hCD40/FcγR mice were injected with 2141 CD40 mAb. After 2.5 hr, blood was analyzed by flow cytometry. Kupffer cells (KCs), macrophages (MFs), Dendritic cells (DCs), conventional type 1 DC (cDC1), conventional type 2 DC (cDC2). Each dot represents an individual mouse, and data are displayed as the mean±SEM. *P≤0.05, **p<0.01, ***P≤0.001, ****P≤0.0001, ns; non-significant.

FIGS. 9A-B show that FcγR-mediated crosslinking is required for CD40/DCs bsAb activity. (A) T cell activation determined by flow cytometry analysis for OVA-specific CD8⁺ T cells in the blood of hCD40/FcγR mice immunized with OVA in the presence of the indicated Fc variants of CD40/DCs bsAbs. Each dot represents an individual mouse. Data are displayed as the mean±SEM. *p≤0.05, **p≤0.01. (B) shows binding of human bsAbs Fc variants to human FcγRIIB Binding of the indicated Fc variants of anti-CD40/DC bsAbs to recombinant hFcγRIIB, assessed by ELISA.

FIGS. 10A-E show that antitumor response by CD40/CD11c bsAb is superior to CD40 mAb when administered at safe doses. (A) Representative H&E staining of livers from hCD40/FcγR mice treated with the indicated doses of CD40 mAb or CD40/CD11c bsAb (n=4 per group). (B) IL-6 and TNF-α secretion following CD40 mAb or CD40/CD11c bsAb treatment. (C) T cell activation determined by flow cytometry analysis of OVA-specific CD8+ T cells in the blood of humanized CD40/FcγR mice immunized with OVA in the presence of the indicated mAb or bsAb. (D) hCD40/FcγR mice were inoculated with MC38 or B16-F10 tumor cells. Once tumors were established and reached an average volume of 50 mm3, mice were treated with CD40 mAb or CD40/CD11c bsAb at their pre-determined MTDs. Tumor volumes were measured via caliper every 3-4 days (n=9-11 per group). (E) hCD40/FcγR mice were inoculated with B16-F10 tumor cells. Mice with established tumors were treated with the indicated mAb/bsAb. Tumor volumes were measured via caliper every 3-4 days (n=9-10 per group). Each dot represents an individual mouse, and data are displayed as the mean±SEM. *P<0.05, **p≤0.01, ***P≤0.001, ****P≤0.0001.

### DESCRIPTION OF SPECIFIC EMBODIMENTS OF THE INVENTION

The present invention, in some embodiments thereof, relates to multispecific antibodies for use in treating diseases.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Therapeutic use of agonistic anti-CD40 monoclonal antibodies (mAbs) is an approach aimed to harness the potency of the immune response to eradicate tumors. This approach has been demonstrated to be effective in many animal models of different tumors. However, human anti CD40 mAbs displayed only modest antitumor activity in patients with solid tumors.

Whilst conceiving embodiments of the present invention, the present inventors have devised a multispecific molecule which targets the CD40 in a dendritic-cells specific manner. Such antibodies having an FcγRIIb binding preference have also been devised.

As is illustrated hereinbelow and in the Examples section which follows, the present inventors have established the cellular pathways that mediate efficacy and toxicity of anti CD40 antibodies. The present inventors have designed a CD40 agonist mAbs having a maximal antitumor activity by lowering the dose-limiting toxicity. The present inventors have designed new type of molecules, in the form of Fc-engineered multispecific (bispecific) antibody, that target both CD40 and a dendritic cell marker. Specifically, a 2141-V11 variant (having enhanced specificity to FcγRIIb) specifically to DCs, the cell population that mediate its antitumor activity, while reducing its binding to CD40 in other cell populations. In order to achieve this specific delivery, the present inventors designed a novel strategy of exploiting the bispecific Abs format as a whole IgG scaffold. Such a design has the advantage of increased specificity toward defined cell populations while maintaining its capability to engage the appropriate FcγR pathway. The present antibodies are endowed with many beneficial properties including (i) defined cellular specificity mediated by dual human Fab recognition and (ii) enhanced FcγRIIB engagement mediated by Fc engineering. To produce the desired bispecific Ab combination it is necessary to correctly assemble two heavy chains and two light chains of two existing Abs. To do so, the present inventors have synthesized the four chains that will form each bsAb. The knob-into-holes technology (identified point mutations in the CH3 domains of the heavy chains) was exploited to enable the heterodimerization of the desired heavy chains [12]. CrossMab technology (exchange of heavy-chain CH1 and light-chain CL1 domains of one of the two Abs composing the bsAb) was applied to ensure the correct association of the light chains and their cognate heavy chains. The variable domains of 2141 were cloned from the previously generated monospecific 2141 Ab into the bispecific Ab constructs. The variable domains of the anti-DEC-205 (HD-109), CD11c (N418), Clec9a, and XRC1 (MARX10) Abs were sequenced and cloned from the HD-109 [13] and N418 [14] hybridomas respectively. The variable domains of Clec9a (10B4), and XRC1 (MARX10) were synthesized based on their sequences described in Patent Application Nos. US20130273150A and EP2641915A1 respectively. The bispecific Ab constructed with the wild type (WT) IgG and the “V11” Fc scaffold (point mutation was introduced using site-directed mutagenesis by PCR) to preserve the optimal high-order cross-linking by FcγRIIB that is necessary for in vivo CD40 activation.

The present inventors have established a synergy between the CD40 binding moiety, the DC targeting and the engagement of FcγRIIB) in vivo, showing optimal anti-tumor activity and minimal toxicity and improved treatment with checkpoint modulation e.g., anti PD-L1.

It is believed that the antibodies of some embodiments of the invention are endowed with improved specificity and therefore therapeutic efficacy and as such can be successfully used in the clinic.

Thus, according to an aspect of the invention there is provided a multispecific antibody comprising a first moiety, which binds and activates CD40 and a second moiety, which specifically binds a dendritic cell (DC).

According to as aspect of the invention there is provided a multispecific antibody comprising a first moiety, which binds and activates CD40, a second moiety, which specifically binds a dendritic cell (DC) and a third moiety comprising a modified Fc region of the multispecific antibody for enhancing specificity and affinity of binding to FcγRIIb.

As used herein a “CD40” refers to “TNF receptor superfamily member 5” (TNFRSF5).

The sequence of human CD40 (NP_001241.1), including 20 amino acid signal sequence, is provided in SEQ ID NO: 41.

CD40 interacts with CD40 ligand (CD40L), which is also referred to as TNFSF5, gp39 and CD154. Unless otherwise indicated, or clear from the context, references to CD40L herein refer to human CD40L (“huCD40L”). Human CD40L is further described in MIM: 300386. The sequence of human CD40L (NP_000065.1) is provided at SEQ ID NO: 42.

It will be appreciated that antibodies to CD40 bind human CD40 and/or mouse CD40. Antibodies that bind both human and mouse are typically referred to as “pan-specific antibodies”.

As mentioned the first moiety binds and activates CD40 (mimicking CD40L) and as such is termed “agonistic”. Agonistic activity can be assayed by testing up-regulation of CD54 or CD86 in human dendritic cells and/or by testing in vivo T cell activation assays (e.g., binding to CD40 is confirmed by ELISA).

First moiety complementary determining sequences (CDRs), which can be used in the multispecific antibody, according to some embodiments of the invention can be found in the antibodies listed hereinbelow:
Anti CD40 2141 (also known as CP870,893) sequences are shown SEQ ID NOs: 5, 6, 11 and 12.

Thus, according to an embodiment of the invention there is provided a multispecific antibody comprising a first moiety comprising complementary determining regions as set forth in SEQ ID NOs: 19-21 in a heavy chain with an N to C orientation and complementary determining regions as set forth in SEQ ID NOs: 22-24 in a light chain with an N to C orientation. CD40 V11 including mutations for optimizing FcγRIIB engagement and bispecific assembly are provided in SEQ ID NOs: 5 and 6 (VH and VL, respectively).
Anti CD40 antibodies: 12D6 and 5F11 are described in WO20170253659, which is herein incorporated by reference in its entirety.
APX005M (Apexigen)—Johnson M, Fakih M, Bendell J, et al. 2017. First in human study with the CD40 agonistic monoclonal antibody APX005M in subjects with solid tumors. J. ImmunoTher. Cancer 5(Suppl. 3):89 (Abstr.)
SGN-40—C.-L. Law et al., “Preclinical antilymphoma activity of a humanized anti-CD40 monoclonal antibody, SGN-40.,” Cancer Res., vol. 65, no. 18, pp. 8331-8338, September 2005.
SEA-CD40—_DOI: 10.1200/JCO.2018.36.15_supp1.3093 Journal of Clinical Oncology 36, no. 15_suppl (May 20, 2018) 3093-3093.

Other CD40 agonistic antibodies are also available in the art.

As mentioned, the multispecific antibody comprises a second moiety which specifically binds to dendritic cells (DCs).

As used herein “specifically” refers to a binding preference to DCs as compared to other cells or platelets, such as cells of the peripheral blood. According to some embodiments, “specifically” means no binding to macrophages (Kupffer or non-Kupffer) and platelets since the target is not expressed on or in a lower density comparing to DCs as determined by flow cytometry analysis.

As used herein, the terms “specific binding,” “selective binding,” “selectively binds,” and “specifically binds,” refer to antibody binding to an epitope on a predetermined antigen but not to other antigens. Typically, the antibody (i) binds with an equilibrium dissociation constant (K_{D}) of approximately less than 10⁻⁷ M, such as approximately less than 10⁻⁸ M, 10⁻⁹ M or 10⁻¹⁰ M or even lower when determined by, e.g., surface plasmon resonance (SPR) technology in a BIACORE®. 2000 surface plasmon resonance instrument using the predetermined antigen, e.g., recombinant DC marker, as the analyte and the antibody as the ligand, or Scatchard analysis of binding of the antibody to antigen positive cells, and (ii) binds to the predetermined antigen with an affinity that is at least two-fold greater than its affinity for binding to a non-specific antigen (e.g., BSA, casein) other than the predetermined antigen or a closely-related antigen. Accordingly, an antibody that “specifically binds to human CD40” or DC marker refers to an antibody that binds to soluble or cell bound human CD40 or DC marker with a K_{D} of 10⁻⁶ M or less, such as approximately less than 10⁻⁷ M, 10⁻⁸ M, 10⁻⁹ M or 10⁻¹⁰ M or even lower.

As used herein “a dendritic cell” (DC) or in plural “dendritic cells” (DCs) refers to cells belonging to a group of cells called professional antigen presenting cells (APCs). DCs have a characteristic morphology, with thin sheets (lamellipodia) extending from the dendritic cell body in several directions. Several phenotypic criteria are also typical, but can vary depending on the source of the dendritic cell. These include high levels of MHC molecules (e.g., class I and class II MHC) and costimulatory molecules (e.g., B7-1 and B7-2), and a lack of markers specific for granulocytes, NK cells, B cells, and T cells. Many dendritic cells express certain markers such as listed below. Dendritic cells are able to initiate primary T cell responses in vitro and in vivo. These responses are antigen specific. Dendritic cells direct a strong mixed leukocyte reaction (MLR) compared to peripheral blood leukocytes, splenocytes, B cells and monocytes. Dendritic cells are optionally characterized by the pattern of cytokine expression by the cell (Zhou and Tedder (1995) Blood 3295-3301). According to a specific embodiment, the multispecific antibody binds immature DCs and possibly mediate they maturation and activation.

According to a specific embodiment, the dendritic cells are cDC1 or cDC2.

According to a specific embodiment, the dendritic cells are cDC1 and cDC2.

According to a specific embodiment, a dendritic cell is characterized by a marker expression selected from the group consisting of wherein said second moiety binds a DC marker selected from the group consisting of CD11c, CD11b, Clec9a, XCR1, DEC-205, BDCA-1, CD8, CD8α, CD103 and MHC-Class II (e.g., HLA-DR), CD141, FLT3, CD13 and CD1c.

According to a specific embodiment, the DCs are human DCs.

According to a specific embodiment, the second moiety binds a DC marker which is selected from the group consisting of CD141, FLT3, CD13, CD1c and HLA-DR (MHC II).

According to a specific embodiment, the second moiety binds a DC marker which is selected from the group consisting CD11c, CD11b, Clec9a, XCR1, DEC-205, BDCA-1, CD8, CD8α, CD103, MHC-Class II (e.g., HLA-DR), CD141, CD13 and CD1c, LI LRA4, LAMPS, CLEC4C, I L3RA and SIGLEC6.

According to a specific embodiment, the DC marker is not LI LRA4, LAMPS, CLEC4C, IL3RA, CLEC9A, XCR1, FLT3, or SIGLEC6.

According to a specific embodiment, the second moiety binds CD11c or DEC-205.

According to a specific embodiment, the second moiety binds CD11c.

Thus, according to an embodiment of the invention there is provided a multispecific antibody comprising a second moiety comprising complementary determining regions as set forth in SEQ ID NOs: 25-27 in a heavy chain with an N to C orientation and complementary determining regions as set forth in SEQ ID NOs: 28-30 in a light chain with an N to C orientation.

According to a specific embodiment, the second moiety binds DEC-205.

Thus, according to an embodiment of the invention there is provided a multispecific antibody comprising a second moiety comprising complementary determining regions as set forth in SEQ ID NOs: 31-33 in a heavy chain with an N to C orientation and complementary determining regions as set forth in SEQ ID NOs: 34-36 in a light chain with an N to C orientation.

According to a specific embodiment, the second moiety binds Clec9a.

Thus, according to an embodiment of the invention there is provided a multispecific antibody comprising a second moiety comprising complementary determining regions as set forth in SEQ ID NOs: 52-54 in a heavy chain with an N to C orientation and complementary determining regions as set forth in SEQ ID NOs: 55-57 in a light chain with an N to C orientation. (CDRs of 10B4).

According to a specific embodiment, the second moiety binds XCR1.

Thus, according to an embodiment of the invention there is provided a multispecific antibody comprising a second moiety comprising complementary determining regions as set forth in SEQ ID NOs: 58-60 in a heavy chain with an N to C orientation and complementary determining regions as set forth in SEQ ID NOs: 61-63 in a light chain with an N to C orientation. (CDRs of MARX10).

Antibodies capable of binding CD11c are well known in the art. N418 is available from ATCC [14].

Antibodies capable of binding DEC-205 are well known in the art. HD-109 is available from Rockeffeler University [13] HD-20, HD-24, HD-71, HD-73, HD-77 and HD-83.

Antibodies capable of binding Clec9a are well known in the art. 10B4 is and others are described in U.S. Patent Application No. US20130273150A [15] (e.g., 1F6, 397, and 7H11 are described in [16]).

Antibodies capable of binding XCR1 are well known in the art. MARX10 is described in EP EP2641915A1 [17].

It will be appreciated that any of the moieties may include Fc modifications that increase binding to FcγRIIB, such as but not limited to, V11 mutations (SEQ ID NO: 2), S267E (“SE”), S267E/L382F (“SELF”), G237D/P238D/P271G/A330R (“V9”), and/or E233D/G237D/P238D/H268D/P271G/A330R (“V12”) corresponding to human IgG1 sequence (positions corresponding to SEQ ID NO: 1).

According to a specific embodiment, the multispecific antibody comprises SEQ ID NOs: 5 and 6 and SEQ ID NOs: 37 and 38.

According to a specific embodiment, the multispecific antibody comprises SEQ ID NOs: 5 and 6 and SEQ ID NOs: 39 and 40.

According to a specific embodiment, the multispecific antibody comprises SEQ ID NOs: 5 and 6 and SEQ ID NOs: 15 and 16.

According to a specific embodiment, the multispecific antibody comprises SEQ ID NOs: 5 and 6 and SEQ ID NOs: 17 and 18.

The term “antibody” as used in this invention includes intact molecules as well as functional fragments thereof (that are capable of binding to an epitope of an antigen).

As used herein, the term “epitope” refers to any antigenic determinant on an antigen to which the paratope of an antibody binds. Epitopic determinants usually consist of chemically active surface groupings of molecules such as amino acids or carbohydrate side chains and usually have specific three dimensional structural characteristics, as well as specific charge characteristics.

According to a specific embodiment, the antibody fragments include, but are not limited to, single chain, Fab, Fab′ and F(ab′)₂ fragments, Fd, Fcab, Fv, dsFv, scFvs, diabodies, minibodies, nanobodies, Fab expression library or single domain molecules such as VH and VL that are capable of binding to an epitope of the antigen in an HLA restricted manner.

Suitable antibody fragments for practicing some embodiments of the invention include a complementarity-determining region (CDR) of an immunoglobulin light chain (referred to herein as “light chain”), a complementarity-determining region of an immunoglobulin heavy chain (referred to herein as “heavy chain”), a variable region of a light chain, a variable region of a heavy chain, a light chain, a heavy chain, an Fd fragment, and antibody fragments comprising essentially whole variable regions of both light and heavy chains such as an Fv, a single chain Fv Fv (scFv), a disulfide-stabilized Fv (dsFv), an Fab, an Fab′, and an F(ab′) 2, or antibody fragments comprising the Fc region of an antibody.

As used herein, the terms “complementarity-determining region” or “CDR” are used interchangeably to refer to the antigen binding regions found within the variable region of the heavy and light chain polypeptides. Generally, antibodies comprise three CDRs in each of the VH (CDR HI or HI; CDR H2 or H2; and CDR H3 or H3) and three in each of the VL (CDR LI or LI; CDR L2 or L2; and CDR L3 or L3).

The identity of the amino acid residues in a particular antibody that make up a variable region or a CDR can be determined using methods well known in the art and include methods such as sequence variability as defined by Kabat et al. (See, e.g., Kabat et al., 1992, Sequences of Proteins of Immunological Interest, 5th ed., Public Health Service, NIH, Washington D.C.), location of the structural loop regions as defined by Chothia et al. (see, e.g., Chothia et al., Nature 342:877-883, 1989.), a compromise between Kabat and Chothia using Oxford Molecular's AbM antibody modeling software (now Accelrys®, see, Martin et al., 1989, Proc. Natl Acad Sci USA. 86:9268; and world wide web site www(dot)bioinf-org(dot)uk/abs), available complex crystal structures as defined by the contact definition (see MacCallum et al., J. Mol. Biol. 262:732-745, 1996) and the “conformational definition” (see, e.g., Makabe et al., Journal of Biological Chemistry, 283:1156-1166, 2008).

As used herein, the “variable regions” and “CDRs” may refer to variable regions and CDRs defined by any approach known in the art, including combinations of approaches.

Functional antibody fragments comprising whole or essentially whole variable regions of both light and heavy chains are defined as follows:
(i) Fv, defined as a genetically engineered fragment consisting of the variable region of the light chain (VL) and the variable region of the heavy chain (VH) expressed as two chains;
(ii) single chain Fv (“scFv”), a genetically engineered single chain molecule including the variable region of the light chain and the variable region of the heavy chain, linked by a suitable polypeptide linker as a genetically fused single chain molecule.
(iii) disulfide-stabilized Fv (“dsFv”), a genetically engineered antibody including the variable region of the light chain and the variable region of the heavy chain, linked by a genetically engineered disulfide bond.
(iv) Fab, a fragment of an antibody molecule containing a monovalent antigen-binding portion of an antibody molecule which can be obtained by treating whole antibody with the enzyme papain to yield the intact light chain and the Fd fragment of the heavy chain which consists of the variable and CH1 domains thereof;
(v) Fab′, a fragment of an antibody molecule containing a monovalent antigen-binding portion of an antibody molecule which can be obtained by treating whole antibody with the enzyme pepsin, followed by reduction (two Fab′ fragments are obtained per antibody molecule);
(vi) F(ab′) 2, a fragment of an antibody molecule containing a monovalent antigen-binding portion of an antibody molecule which can be obtained by treating whole antibody with the enzyme pepsin (i.e., a dimer of Fab′ fragments held together by two disulfide bonds);
(vii) Single domain antibodies or nanobodies are composed of a single VH or VL domains which exhibit sufficient affinity to the antigen; and
(viii) Fcab, a fragment of an antibody molecule containing the Fc portion of an antibody developed as an antigen-binding domain by introducing antigen-binding ability into the Fc region of the antibody.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Exemplary methods for generating antibodies employ induction of in-vivo production of antibody molecules, screening of immunoglobulin libraries (Orlandi D. R. et al., 1989. Proc. Natl. Acad. Sci. U.S.A 86:3833-3837; Winter G. et al., 1991. Nature 349:293-299) or generation of monoclonal antibody molecules by continuous cell lines in culture. These include, but are not limited to, the hybridoma technique, the human B-cell hybridoma technique, and the Epstein-Barr virus (EBV)-hybridoma technique (Kohler G. et al., 1975. Nature 256:495-497; Kozbor D. et al., 1985. J. Immunol. Methods 81:31-42; Cote R J. et al., 1983. Proc. Natl. Acad. Sci. U.S.A 80:2026-2030; Cole S P. et al., 1984. Mol. Cell. Biol. 62:109-120).

In cases where target antigens are too small to elicit an adequate immunogenic response when generating antibodies in-vivo, such antigens (haptens) can be coupled to antigenically neutral carriers such as keyhole limpet hemocyanin (KLH) or serum albumin [e.g., bovine serum albumine (BSA)] carriers (see, for example, U.S. Pat. Nos. 5,189,178 and 5,239,078]. Coupling a hapten to a carrier can be effected using methods well known in the art. For example, direct coupling to amino groups can be effected and optionally followed by reduction of the imino linkage formed. Alternatively, the carrier can be coupled using condensing agents such as dicyclohexyl carbodiimide or other carbodiimide dehydrating agents. Linker compounds can also be used to effect the coupling; both homobifunctional and heterobifunctional linkers are available from Pierce Chemical Company, Rockford, Ill. The resulting immunogenic complex can then be injected into suitable mammalian subjects such as mice, rabbits, and the like. Suitable protocols involve repeated injection of the immunogen in the presence of adjuvants according to a schedule which boosts production of antibodies in the serum. The titers of the immune serum can readily be measured using immunoassay procedures which are well known in the art.

The antisera obtained can be used directly or monoclonal antibodies may be obtained as described hereinabove.

Antibody fragments according to some embodiments of the invention can be prepared by proteolytic hydrolysis of the antibody or by expression in E. coli or mammalian cells (e.g. Chinese hamster ovary cell culture or other protein expression systems) of DNA encoding the fragment.

Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

As described hereinabove, Fv fragments comprise an association of VH and VL chains. This association may be noncovalent, as described in Inbar et al. [Proc. Nat'l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by [Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

As mentioned, the antibody fragment may comprise a Fc region of an antibody termed “Fcab”. Such antibody fragments typically comprise the CH2-CH3 domains of an antibody. Fcabs are engineering to comprise at least one modification in a structural loop region of the antibody, i.e. in a CH3 region of the heavy chain. Such antibody fragments can be generated, for example, as follows: providing a nucleic acid encoding an antibody comprising at least one structural loop region (e.g. Fc region), modifying at least one nucleotide residue of the at least one structural loop regions, transferring the modified nucleic acid in an expression system, expressing the modified antibody, contacting the expressed modified antibody with an epitope, and determining whether the modified antibody binds to the epitope. See, for example, U.S. Pat. Nos. 9,045,528 and 9,133,274 incorporated herein by reference in their entirety.

Humanized forms of non-human (e.g., murine) antibodies are chimeric molecules of immunoglobulins, immunoglobulin chains or fragments thereof (such as Fv, Fab, Fab′, F(ab′).sub.2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10,: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

Unless otherwise indicated, an immunoglobulin may be from any of the commonly known isotypes, including but not limited to IgA, secretory IgA, IgG and IgM. The IgG isotype is divided in subclasses in certain species: IgG1, IgG2, IgG3 and IgG4 in humans, and IgG1, IgG2a, IgG2b and IgG3 in mice. Immunoglobulins, e.g., human IgG1, exist in several allotypes, which differ from each other in at most a few amino acids.

According to a specific embodiment, the antibody is of an IgG1 isotype. Once antibodies are obtained, they may be tested for activity, for example via ELISA, Western blotting, FACS, dot blot and any other method for antibody qualification.

According to a specific embodiment, the antibody is trifunctional in its binding to Fey, CD40 and DCs.

As used herein a “multispecific antibody” is an antibody that can bind simultaneously to at least two targets that are of different structure, two different antigens or two different epitopes one on CD40 and at least one another on a DC, as mentioned.

Specificity indicates how many antigens or epitopes an antibody is able to bind; i.e., bispecific, trispecific, quatraspecific. According to a specific embodiment, the antibody is a bispecific antibody.

Using these definitions, a natural antibody, e.g., an IgG, is bivalent because it has two binding arms but is monospecific because it binds to one epitope.

A “bispecific antibody” is an antibody that can bind simultaneously to two targets which are of different structure, one on CD40 and at least one another on a DC.

Valency indicates how many binding arms or sites the antibody has to a single antigen or epitope; i.e., monovalent, bivalent, trivalent or multivalent. The multivalency of the antibody means that it can take advantage of multiple interactions in binding to an antigen, thus increasing the avidity of binding to the antigen.

Multispecific, multivalent antibodies are constructs that have more than one binding site of different specificity. For example, a diabody, where one binding site reacts with one antigen and the other with another antigen.

As used herein, a “moiety” refers to an antibody component of the multispecific (e.g., bispecific) antibody capable of binding the indicated target.

In order to produce the multispecific antibody of some embodiments of the invention, the present moieties can be modified at the Fc region e.g., the CH3 domain (according to kabat) as well known in the art. Such a modification ensures correct assembly of the multispecific antibody via the heavy chains.

Accordingly, the CH3 domain of one heavy chain is altered, so that within the original interface the CH3 domain of one heavy chain that meets the original interface of the CH3 domain of the other heavy chain within the multispecific antibody, an amino acid residue is replaced with an amino acid residue having a larger side chain volume, thereby generating a protuberance within the interface of the CH3 domain of one heavy chain which is positionable in a cavity within the interface of the CH3 domain of the other heavy chain; and the CH3 domain of the other heavy chain is altered, so that within the original interface of the second CH3 domain that meets the original interface of the first CH3 domain within the trivalent, bispecific antibody an amino acid residue is replaced with an amino acid residue having a smaller side chain volume, thereby generating a cavity within the interface of the second CH3 domain within which a protuberance within the interface of the first CH3 domain is positionable (also known as “the knobs-into-holes” approach by Genentech).

According to a specific embodiment, the amino acid residue having a larger side chain volume is selected from the group consisting of arginine (R), phenylalanine (F), tyrosine (Y), tryptophan (W).

According to a specific embodiment, the amino acid residue having a smaller side chain volume is selected from the group consisting of alanine (A), serine (S), threonine (T), valine (V).

According to a specific embodiment, both CH3 domains are further altered by the introduction of cysteine (C) as amino acid in the corresponding positions of each CH3 domain such that a disulfide bridge between both CH3 domains can be formed.

In a specific embodiment, the bispecific comprises a T366W mutation in the CH3 domain of the “knobs chain” and T366S, L368A, Y407V mutations in the CH3 domain of the “hole chain”. An additional interchain disulfide bridge between the CH3 domains can also be used (Merchant, A. M., et al., Nature Biotech 16 (1998) 677-681) e.g. by introducing a Y349C mutation into the CH3 domain of the “knobs chain” and a E356C mutation or a S354C mutation into the CH3 domain of the “hole chain”. Thus in a another preferred embodiment, the bispecific antibody comprises Y349C, T366W mutations in one of the two CH3 domains and E356C, T366S, L368A, Y407V mutations in the other of the two CH3 domains or the bispecific antibody comprises Y349C, T366W mutations in one of the two CH3 domains and S354C, T366S, L368A, Y407V mutations in the other of the two CH3 domains (the additional Y349C mutation in one CH3 domain and the additional E356C or S354C mutation in the other CH3 domain forming a interchain disulfide bridge) (numbering always according to EU index of Kabat). But also other knobs-in-holes technologies as described by EP 1 870 459A1, can be used alternatively or additionally. A specific example for the bispecific antibody are R409D; K370E mutations in the CH3 domain of the “knobs chain” and D399K; E357K mutations in the CH3 domain of the “hole chain” (numbering always according to EU index of Kabat).

In another embodiment the bispecific antibody comprises a T366W mutation in the CH3 domain of the “knobs chain” and T366S, L368A, Y407V mutations in the CH3 domain of the “hole chain” and additionally R409D; K370E mutations in the CH3 domain of the “knobs chain” and D399K; E357K mutations in the CH3 domain of the “hole chain”.

In another embodiment the bispecific antibody comprises Y349C, T366W mutations in one of the two CH3 domains and S354C, T366S, L368A, Y407V mutations in the other of the two CH3 domains or the bispecific antibody comprises Y349C, T366W mutations in one of the two CH3 domains and S354C, T366S, L368A, Y407V mutations in the other of the two CH3 domains and additionally R409D; K370E mutations in the CH3 domain of the “knobs chain” and D399K; E357K mutations in the CH3 domain of the “hole chain”.

According to a specific embodiment, Y349C/T366S/L368A/Y407V mutations are introduced for the 1st mAb (e.g., anti DC) and S354C/T366W for the 2nd mAb (e.g., anti CD40) (Merchant et al., 1998; Ridgway et al., 1996).

Alternatively or additionally, for correct heavy-light chain pairing, at least one of the moieties can be expressed in the CrossMab format (CH1-CL swapping).

The basis of the CrossMab technology is the crossover of antibody domains within one arm of a bispecific IgG antibody enabling correct chain association, whereas correct heterodimerization of the heavy chains can be achieved by the knob-into-hole technology as described above or charge interactions. This can be achieved by exchange of different domains within a Fab-fragment. Either the Fab domains (in the CrossMab^{Fab} format), or only the variable VH-VL domains (CrossMab^{VH-VL} format) or the constant CH1-CL domains (CrossMab^{CH1-CL} format) within the Fab-fragment can be exchanged for this purpose. Indeed, for the CrossMab^{CH1-CL} format the respective original light chain and the novel VL-CH1 light chain do not result in undesired interactions with the respective original and VH-CL containing heavy chains, and no theoretical side products can be formed. In contrast, in the case of the CrossMab^{Fab} format a non-functional monovalent antibody (MoAb) as well as a non-functional Fab-fragment can be formed. These side products can be removed by chromatographic techniques. In the case of the CrossMab^{VH-VL} format an undesired side product with a VL-CH1/VL-CL domain association known from Bence-Jones proteins can occur between the VL-CH1 containing heavy chain and the original unmodified VL-CL light chain. The introduction of repulsive charge pairs based on existing conserved charge pairs in the wildtype antibody framework into the constant CH1 and CL domains of the wildtype non-crossed Fab-fragment can overcome the formation of this Bence-Jones-like side product in the CrossMab^{VH-VL+/-} format. More details on CrossMab Technology can be found in Klein et al. Methods 154, 1 Feb. 2019, Pages 21-31c.

Alternatively, multispecific e.g., bispecific antibodies described herein can be prepared by conjugating the moieties using methods known in the art. For example, each moiety of the multispecific antibody can be generated separately and then conjugated to one another. A variety of coupling or cross-linking agents can be used for covalent conjugation. Examples of cross-linking agents include protein A, carbodiimide, N-succinimidyl-S-acetyl-thioacetate (SATA), 5,5′-dithiobis(2-nitrobenzoic acid) (DTNB), o-phenylenedimaleimide (oPDM), N-succinimidyl-3-(2-pyridyldithio)propionate (SPDP), and sulfosuccinimidyl 4-(N-maleimidomethyl) cyclohaxane-1-carboxylate (sulfo-SMCC) (see e.g., Karpovsky et al. (1984) J. Exp. Med. 160:1686; Liu, M A et al. (1985) Proc. Natl. Acad. Sci. (USA) 82:8648). Other methods include those described in Paulus (1985) Behring Ins. Mitt. No. 78, 118-132; Brennan et al. (1985) Science 229:81-83), and Glennie et al. (1987) J. Immunol. 139: 2367-2375). Preferred conjugating agents are SATA and sulfo-SMCC, both available from Pierce Chemical Co. (Rockford, Ill.).

Alternatively or additionally, the conjugation of each moiety of the multispecific antibody can be done via sulfhydryl bonding of the C-terminus hinge regions of the two heavy chains. In a specific embodiment, the hinge region is modified to contain an odd number of sulfhydryl residues, preferably one, prior to conjugation.

According to a specific embodiment, a third moiety conferring enhanced specificity to FcγRIIb receptor.

An “Fc receptor” or “FcR” is a receptor that binds to the Fc region of an immunoglobulin. FcRs that bind to an IgG antibody comprise receptors of the FcγR family, including allelic variants and alternatively spliced forms of these receptors. The FcγR family consists of three activating (FcγRI, FcγRIII, and FcγRIV in mice; FcγRIA, FcγRIIA, and FcγRIIIA in humans) and one inhibitory (FcγRIIb, or equivalently RcγRIIB) receptor. Various properties of human FcγRs are summarized in US20170253659 (and Table 1 therein).

The majority of innate effector cell types co-express one or more activating FcγR and the inhibitory FcγRIIb, whereas natural killer (NK) cells selectively express one activating Fc receptor (FcγRIII in mice and FcγRIIIA in humans) but not the inhibitory FcγRIIb in mice and humans. Human IgG1 binds to most human Fc receptors and is considered equivalent to murine IgG2a with respect to the types of activating Fc receptors that it binds to.

Human FcγRIIB have low affinity to human IgG. As mentioned hereinabove, a number of previous publications demonstrated that engagement of the inhibitory FcγRIIB is an absolute requirement for the in-vivo antitumor activity of agonistic Abs targeting mouse CD40, as well as other members of the TNFR family. This is due to the high-order crosslinking of the CD40 antibody by FcγRIIB expressed on neighboring cells. Such crosslinking enhances the clustering of CD40 on the cell surface and results in enhanced CD40 signaling [6][7].

Thus according to an embodiment of the invention, the multispecific antibody comprises a third moiety comprising a modified Fc region of the multispecific antibody for enhancing specificity and affinity of binding to FcγRIIb.

The modified (mutant) Fc region has one or more mutations corresponding to one or more mutations in a human IgG heavy chain (SEQ ID NO: 1) selected from the group consisting of N297A, S267E (“SE”), S267E/L382F (“SELF”), G237D/P238D/P271G/A330R (“V9”), or G237D/P238D/H268D/P271G/A330R (“V11”) (SEQ ID NO:2), or (“V12”).

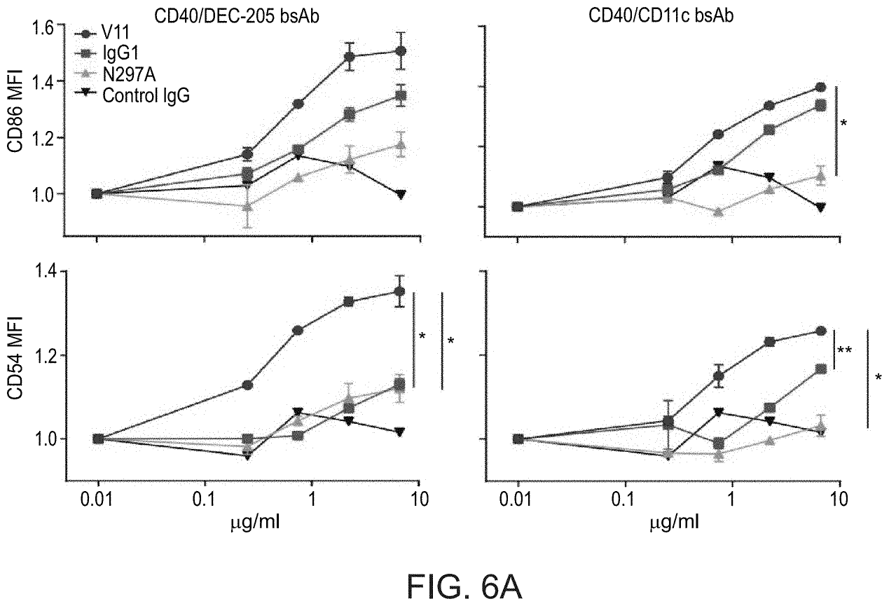

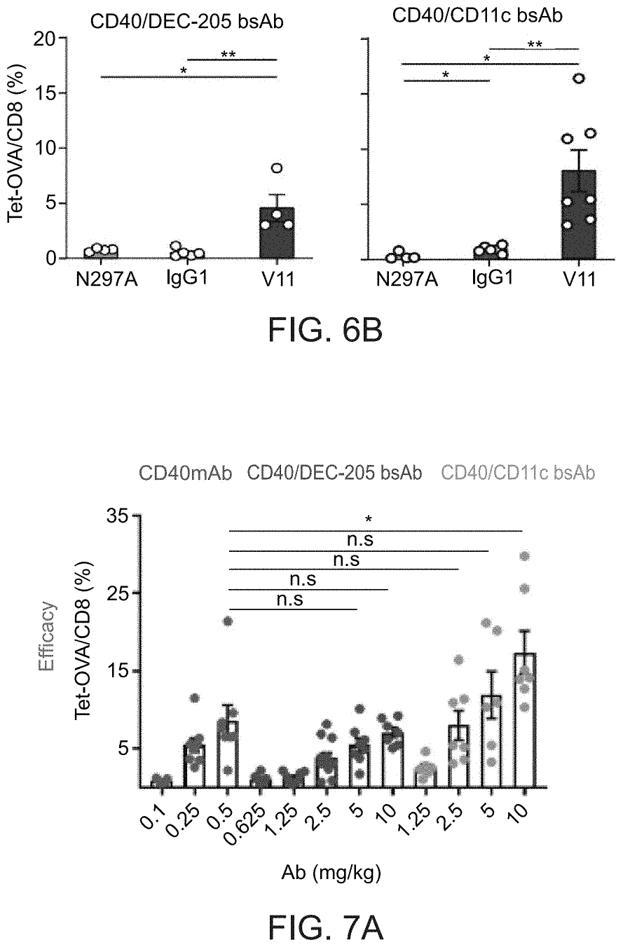

According to a specific embodiment, the modified Fc is of the V11 mutant. As shown in the Examples section which follows, in-vitro and in-vivo experiments showed that multispecific antibodies benefit from FcγRIIB engagement in order to effectively activate DCs and T-cells (FIG. 6A-B).

Another aspect described herein pertains to nucleic acid molecules that encode the antibodies described herein. The nucleic acids may be present in whole cells, in a cell lysate, or in a partially purified or substantially pure form. A nucleic acid is “isolated” or “rendered substantially pure” when purified away from other cellular components or other contaminants, e.g., other cellular nucleic acids (e.g., other chromosomal DNA, e.g., the chromosomal DNA that is linked to the isolated DNA in nature) or proteins, by standard techniques, including alkaline/SDS treatment, CsCl banding, column chromatography, restriction enzymes, agarose gel electrophoresis and others well known in the art. See, F. Ausubel, et al, ed. (1987) Current Protocols in Molecular Biology, Greene Publishing and Wiley Interscience, New York. A nucleic acid described herein can be, for example, DNA or RNA and may or may not contain intronic sequences. In a certain embodiments, the nucleic acid is a cDNA molecule.

Nucleic acids described herein can be obtained using standard molecular biology techniques. For antibodies expressed by hybridomas {e.g., hybridomas prepared from transgenic mice carrying human immunoglobulin genes as described further below), cDNAs encoding the light and heavy chains of the antibody made by the hybridoma can be obtained by standard PCR amplification or cDNA cloning techniques. For antibodies obtained from an immunoglobulin gene library {e.g., using phage display techniques), nucleic acid encoding the antibody can be recovered from the library.

Once DNA fragments encoding V_{H} and V_{L} segments are obtained, these DNA fragments can be further manipulated by standard recombinant DNA techniques, for example to convert the variable region genes to full-length antibody chain genes, to Fab fragment genes or to a scFv gene. In these manipulations, a V_{L}- or V_{H}-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame.

The isolated DNA encoding the VH region can be converted to a full-length heavy chain gene by operatively linking the VH-encoding DNA to another DNA molecule encoding heavy chain constant regions (hinge, CHI, CH2 and/or CH3). The sequences of human heavy chain constant region genes are known in the art (see e.g., Kabat, E. A., et al. (1991) Sequences of Proteins of Immunological Interest, Fifth Edition, U.S.

Department of Health and Human Services, NIH Publication No. 91-3242) and DNA fragments encompassing these regions can be obtained by standard PCR amplification. The heavy chain constant region can be an IgGl, IgG2, IgG3, IgG4, IgA, IgE, IgM or IgD constant region, for example, an IgG1 region. For a Fab fragment heavy chain gene, the VH-encoding DNA can be operatively linked to another DNA molecule encoding only the heavy chain CHI constant region.

The isolated DNA encoding the V_{L} region can be converted to a full-length light chain gene (as well as a Fab light chain gene) by operatively linking the VL-encoding DNA to another DNA molecule encoding the light chain constant region, CL. The sequences of human light chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The light chain constant region can be a kappa or lambda constant region.

A variety of prokaryotic or eukaryotic cells can be used as host-expression systems to express the antibodies of some embodiments of the invention. These include, but are not limited to, microorganisms, such as bacteria transformed with a recombinant bacteriophage DNA, plasmid DNA or cosmid DNA expression vector containing the coding sequence; yeast transformed with recombinant yeast expression vectors containing the coding sequence; plant cell systems infected with recombinant virus expression vectors (e.g., cauliflower mosaic virus, CaMV; tobacco mosaic virus, TMV) or transformed with recombinant plasmid expression vectors, such as Ti plasmid, containing the coding sequence. Mammalian expression systems can also be used to express the antibodies of some embodiments of the invention. Conditions of expression in culture depend on the expression system used.

Recovery of the antibody from the culture is effected following an appropriate time in the culture. The phrase “recovering the recombinant antibody” refers to collecting the whole fermentation medium containing the antibody and need not imply additional steps of separation or purification. Notwithstanding the above, antibodies of some embodiments of the invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization.

The antibodies, antibody compositions and methods described herein have numerous in vitro and in vivo utilities involving, for example, enhancement of immune response by agonizing CD40 signaling. In a preferred embodiment, the antibodies described herein are human or humanized antibodies. For example, multispecific antibodies described herein can be administered to cells in culture, in vitro or ex vivo, or to human subjects, e.g., in vivo, to enhance immunity in a variety of diseases.

Accordingly, provided herein are methods of modifying an immune response in a subject comprising administering to the subject the multispecific antibody, described herein such that the immune response in the subject is enhanced, stimulated or up-regulated.

As used herein, the term “subject” includes mammals, such as human beings at any age which suffer from a disorder, e.g., cancer, chronic viral infection. According to a specific embodiment, this term encompasses individuals who are at risk to develop the disorder.

According to a specific embodiment, subjects include human patients in whom enhancement of an immune response would be desirable. The methods are particularly suitable for treating human patients having a disorder that can be treated by augmenting an immune response (e.g., the T-cell mediated immune response). In a particular embodiment, the methods are particularly suitable for treatment of cancer. To achieve antigen-specific enhancement of immunity, the multispecific antibodies described herein can be administered together with an antigen of interest or the antigen may already be present in the subject to be treated (e.g., a tumor-bearing or virus-bearing subject). When antibodies to CD40 are administered together with another agent, the two can be administered separately or simultaneously.

Further encompassed are methods of enhancing an immune response (e.g., T cell mediated anti tumor immunity) in a subject comprising administering multispecific antibodies described herein to the subject such that an immune response in the subject is enhanced. In a preferred embodiment, the subject is a tumor-bearing subject and an immune response against the tumor is enhanced. A tumor may be a solid tumor or a liquid tumor, e.g., a hematological malignancy. In certain embodiments, a tumor is an immunogenic tumor. In certain embodiments, a tumor is non-immunogenic. In certain embodiments, a tumor is PD-L1 positive. In certain embodiments a tumor is PD-L1 negative. A subject may also be a virus-bearing subject and an immune response against the virus is enhanced.

Further provided are methods for inhibiting growth of tumor cells in a subject comprising administering to the subject the multispecific antibodies described herein such that growth of the tumor is inhibited in the subject. Also provided are methods of treating chronic viral infection in a subject comprising administering to the subject multispecific antibodies described herein such that the chronic viral infection is treated in the subject.

In certain embodiments, multispecific antibodies described herein are given to a subject as an adjunctive therapy. Treatments of subjects having cancer with multispecific antibodies described herein may lead to a long-term durable response relative to the current standard of care; long term survival of at least 1, 2, 3, 4, 5, 10 or more years, recurrence free survival of at least 1, 2, 3, 4, 5, or 10 or more years. In certain embodiments, treatment of a subject having cancer with multispecific antibodies described herein prevents recurrence of cancer or delays recurrence of cancer by, e.g., 1, 2, 3, 4, 5, or 10 or more years. An anti-CD40 treatment can be used as a primary or secondary line of treatment.

Provided herein are methods for treating a subject having cancer, comprising administering to the subject the multispecific antibodies described herein, such that the subject is treated, e.g., such that growth of cancerous tumors is inhibited or reduced and/or that the tumors regress. Multispecific antibodies described herein can be used alone to inhibit the growth of cancerous tumors. Alternatively, multispecific antibodies described herein can be used in conjunction with another agent, e.g., other immunogenic agents, standard cancer treatments, or other antibodies, as described below.

Accordingly, provided herein are methods of treating cancer, e.g., by inhibiting growth of tumor cells, in a subject, comprising administering to the subject a therapeutically effective amount of the multispecific antibodies described herein.

Cancers whose growth may be inhibited using the antibodies of the invention include cancers typically responsive to immunotherapy. Non-limiting examples of cancers for treatment include squamous cell carcinoma, small-cell lung cancer, non-small cell lung cancer, squamous non-small cell lung cancer (NSCLC), non NSCLC, glioma, gastrointestinal cancer, renal cancer (e.g. clear cell carcinoma), ovarian cancer, liver cancer, colorectal cancer, endometrial cancer, kidney cancer (e.g., renal cell carcinoma (RCC)), prostate cancer (e.g. hormone refractory prostate adenocarcinoma), thyroid cancer, neuroblastoma, pancreatic cancer, glioblastoma (glioblastoma multiforme), cervical cancer, stomach cancer, bladder cancer, hepatoma, breast cancer, colon carcinoma, and head and neck cancer (or carcinoma), gastric cancer, germ cell tumor, pediatric sarcoma, sinonasal natural killer, melanoma (e.g., metastatic malignant melanoma, such as cutaneous or intraocular malignant melanoma), bone cancer, skin cancer, uterine cancer, cancer of the anal region, testicular cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the parathyroid gland, cancer of the adrenal gland, sarcoma of soft tissue, cancer of the urethra, cancer of the penis, solid tumors of childhood, cancer of the ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally-induced cancers including those induced by asbestos, virus-related cancers (e.g., human papilloma virus (HPV)-related tumor), and hematologic malignancies derived from either of the two major blood cell lineages, i.e., the myeloid cell line (which produces granulocytes, erythrocytes, thrombocytes, macrophages and mast cells) or lymphoid cell line (which produces B, T, NK and plasma cells), such as all types of leukemias, lymphomas, and myelomas, e.g., acute, chronic, lymphocytic and/or myelogenous leukemias, such as acute leukemia (ALL), acute myelogenous leukemia (AML), chronic lymphocytic leukemia (CLL), and chronic myelogenous leukemia (CML), undifferentiated AML (MO), myeloblastic leukemia (M1), myeloblastic leukemia (M2; with cell maturation), promyelocytic leukemia (M3 or M3 variant [M3V]), myelomonocytic leukemia (M4 or M4 variant with eosinophilia [M4E]), monocytic leukemia (M5), erythroleukemia (M6), megakaryoblastic leukemia (M7), isolated granulocytic sarcoma, and chloroma; lymphomas, such as Hodgkin's lymphoma (HL), non-Hodgkin's lymphoma (NHL), B-cell lymphomas, T-cell lymphomas, lymphoplasmacytoid lymphoma, monocytoid B-cell lymphoma, mucosa-associated lymphoid tissue (MALT) lymphoma, anaplastic (e.g., Ki 1+) large-cell lymphoma, adult T-cell lymphoma/leukemia, mantle cell lymphoma, angio immunoblastic T-cell lymphoma, angiocentric lymphoma, intestinal T-cell lymphoma, primary mediastinal B-cell lymphoma, precursor T-lymphoblastic lymphoma, T-lymphoblastic; and lymphoma/leukemia (T-Lbly/T-ALL), peripheral T-cell lymphoma, lymphoblastic lymphoma, post-transplantation lymphoproliferative disorder, true histiocytic lymphoma, primary central nervous system lymphoma, primary effusion lymphoma, lymphoblastic lymphoma (LBL), hematopoietic tumors of lymphoid lineage, acute lymphoblastic leukemia, diffuse large B-cell lymphoma, Burkitt's lymphoma, follicular lymphoma, diffuse histiocytic lymphoma (DHL), immunoblastic large cell lymphoma, precursor B-lymphoblastic lymphoma, cutaneous T-cell lymphoma (CTLC) (also called mycosis fungoides or Sezary syndrome), and lymphoplasmacytoid lymphoma (LPL) with Waldenstrom's macroglobulinemia; myelomas, such as IgG myeloma, light chain myeloma, nonsecretory myeloma, smoldering myeloma (also called indolent myeloma), solitary plasmocytoma, and multiple myelomas, chronic lymphocytic leukemia (CLL), hairy cell lymphoma; hematopoietic tumors of myeloid lineage, tumors of mesenchymal origin, including fibrosarcoma and rhabdomyoscarcoma; seminoma, teratocarcinoma, tumors of the central and peripheral nervous, including astrocytoma, schwannomas; tumors of mesenchymal origin, including fibrosarcoma, rhabdomyoscaroma, and osteosarcoma; and other tumors, including melanoma, xeroderma pigmentosum, keratoacanthoma, seminoma, thyroid follicular cancer and teratocarcinoma, hematopoietic tumors of lymphoid lineage, for example T-cell and B-cell tumors, including but not limited to T-cell disorders such as T-prolymphocytic leukemia (T-PLL), including of the small cell and cerebriform cell type; large granular lymphocyte leukemia (LGL) preferably of the T-cell type; a/d T-NHL hepatosplenic lymphoma; peripheral/post-thymic T cell lymphoma (pleomorphic and immunoblastic subtypes); angiocentric (nasal) T-cell lymphoma; cancer of the head or neck, renal cancer, rectal cancer, cancer of the thyroid gland; acute myeloid lymphoma, as well as any combinations of said cancers. The methods described herein may also be used for treatment of metastatic cancers, refractory cancers (e.g., cancers refractory to previous immunotherapy, e.g., with a blocking CTLA-4 or PD-1 antibody), and recurrent cancers.

According to a specific embodiment, the cancer is selected from the group consisting of: bladder cancer, breast cancer, uterine/cervical cancer, ovarian cancer, prostate cancer, testicular cancer, esophageal cancer, gastrointestinal cancer, pancreatic cancer, colorectal cancer, colon cancer, kidney cancer, head and neck cancer, lung cancer, stomach cancer, germ cell cancer, bone cancer, liver cancer, thyroid cancer, skin cancer, neoplasm of the central nervous system, lymphoma, leukemia, myeloma, sarcoma, and virus-related cancer.

the multispecific antibodies described herein can be administered as a monotherapy, or as the only immunostimulating therapy, or it can be combined with an immunogenic agent in a cancer vaccine strategy, such as cancerous cells, purified tumor antigens (including recombinant proteins, peptides, and carbohydrate molecules), cells, and cells transfected with genes encoding immune stimulating cytokines (He et al. (2004) J. Immunol. 173:4919-28). Non-limiting examples of tumor vaccines that can be used include peptides of melanoma antigens, such as peptides of gp100, MAGE antigens, Trp-2, MART1 and/or tyrosinase, or tumor cells transfected to express the cytokine GM-CSF. Many experimental strategies for vaccination against tumors have been devised (see Rosenberg, S., 2000, Development of Cancer Vaccines, ASCO Educational Book Spring: 60-62; Logothetis, C., 2000, ASCO Educational Book Spring: 300-302; Khayat, D. 2000, ASCO Educational Book Spring: 414-428; Foon, K. 2000, ASCO Educational Book Spring: 730-738; see also Restifo, N. and Sznol, M., Cancer Vaccines, Ch. 61, pp. 3023-3043 in DeVita et al. (eds.), 1997, Cancer: Principles and Practice of Oncology, Fifth Edition). In one of these strategies, a vaccine is prepared using autologous or allogeneic tumor cells. These cellular vaccines have been shown to be most effective when the tumor cells are transduced to express GM-CSF. GM-CSF has been shown to be a potent activator of antigen presentation for tumor vaccination. Dranoff et al. (1993) Proc. Natl. Acad. Sci. (USA) 90: 3539-43.

Other tumor vaccines can include the proteins from viruses implicated in human cancers such a Human Papilloma Viruses (HPV), Hepatitis Viruses (HBV and HCV) and Kaposi's Herpes Sarcoma Virus (KHSV). Another form of tumor specific antigen that can be used in conjunction with CD40 activation is purified heat shock proteins (HSP) isolated from the tumor tissue itself. These heat shock proteins contain fragments of proteins from the tumor cells and these HSPs are highly efficient at delivery to antigen presenting cells for eliciting tumor immunity (Suot & Srivastava (1995) Science 269:1585-1588; Tamura et al. (1997) Science 278:117-120).

Dendritic cells (DC) are potent antigen presenting cells that can be used to prime antigen-specific responses. DC's can be produced ex vivo and loaded with various protein and peptide antigens as well as tumor cell extracts (Nestle et al. (1998) Nature Medicine 4: 328-332). DCs can also be transduced by genetic means to express these tumor antigens as well. DCs have also been fused directly to tumor cells for the purposes of immunization (Kugler et al. (2000) Nature Medicine 6:332-336). As a method of vaccination, DC immunization can be effectively combined with CD40 agonism to activate (unleash) more potent anti-tumor responses. The multispecific antibodies described herein can also be combined with standard cancer treatments (e.g., surgery, radiation, and chemotherapy). Agonism of CD40 can be effectively combined with chemotherapeutic regimes. In these instances, it may be possible to reduce the dose of chemotherapeutic reagent administered (Mokyr et al. (1998) Cancer Research 58: 5301-5304). An example of such a combination is an anti-huCD40 antibody in combination with decarbazine for the treatment of melanoma. Another example of such a combination is the multispecific antibodies described herein in combination with interleukin-2 (IL-2) for the treatment of melanoma. The scientific rationale behind the combined use of CD40 agonists and chemotherapy is that cell death, that is a consequence of the cytotoxic action of most chemotherapeutic compounds, should result in increased levels of tumor antigen in the antigen presentation pathway. Other combination therapies that may result in synergy with CD40 agonism through cell death are radiation, surgery, and hormone deprivation. Each of these protocols creates a source of tumor antigen in the host. Angiogenesis inhibitors can also be combined with CD40 agonists. Inhibition of angiogenesis leads to tumor cell death which may feed tumor antigen into host antigen presentation pathways.

Tumors evade host immune surveillance by a large variety of mechanisms. Many of these mechanisms may be overcome by the inactivation of immunosuppressive proteins expressed by the tumors. These include among others TGF-.beta. (Kehrl et al. (1986) J. Exp. Med. 163: 1037-1050), IL-10 (Howard & O'Garra (1992) Immunology Today 13: 198-200), and Fas ligand (Hahne et al. (1996) Science 274: 1363-1365). Antibodies to each of these entities can be used in combination with anti-huCD40 antibodies to counteract the effects of the immunosuppressive agent and favor tumor immune responses by the host.

The multispecific antibodies described herein are able to substitute effectively for T cell helper activity. Ridge et al. (1998) Nature 393: 474-478. Activating antibodies to T cell costimulatory molecules such as CTLA-4 (e.g., U.S. Pat. No. 5,811,097), OX-40 (Weinberg et al. (2000) Immunol 164: 2160-2169), CD137/4-1BB (Melero et al. (1997) Nature Medicine 3: 682-685 (1997), and ICOS (Hutloff et al. (1999) Nature 397: 262-266) may also provide for increased levels of T cell activation. Inhibitors of PD1 or PD-L1 may also be used in conjunction with the multispecific antibodies described herein.

There are also several experimental treatment protocols that involve ex vivo activation and expansion of antigen specific T cells and adoptive transfer of these cells into recipients in order to stimulate antigen-specific T cells against tumor (Greenberg & Riddell (1999) Science 285: 546-51). These methods can also be used to activate T cell responses to infectious agents such as CMV. Ex vivo activation in the presence of the multispecific antibodies described herein can increase the frequency and activity of the adoptively transferred T cells.

In another aspect, the invention described herein provides a method of treating an infectious disease in a subject comprising administering to the subject the multispecific antibodies described herein, such that the subject is treated for the infectious disease.

Similar to its application to tumors as discussed above, antibody-mediated CD40 agonism can be used alone, or as an adjuvant, in combination with vaccines, to enhance the immune response to pathogens, toxins, and self-antigens. Examples of pathogens for which this therapeutic approach can be particularly useful, include pathogens for which there is currently no effective vaccine, or pathogens for which conventional vaccines are less than completely effective. These include, but are not limited to HIV, Hepatitis (A, B, & C), Influenza, Herpes, Giardia, Malaria, Leishmania, Staphylococcus aureus, Pseudomonas aeruginosa. CD40 agonism is particularly useful against established infections by agents such as HIV that present altered antigens over the course of the infections. These novel epitopes are recognized as foreign at the time of anti-human CD40 antibody administration, thus provoking a strong T cell response.

Some examples of pathogenic viruses causing infections treatable by methods described herein include HIV, hepatitis (A, B, or C), herpes virus (e.g., VZV, HSV-1, HAV-6, HSV-II, and CMV, Epstein Barr virus), adenovirus, influenza virus, flaviviruses, echovirus, rhinovirus, coxsackie virus, coronavirus, respiratory syncytial virus, mumps virus, rotavirus, measles virus, rubella virus, parvovirus, vaccinia virus, HTLV virus, dengue virus, papillomavirus, molluscum virus, poliovirus, rabies virus, JC virus and arboviral encephalitis virus.

Some examples of pathogenic bacteria causing infections treatable by methods described herein include chlamydia, rickettsial bacteria, mycobacteria, staphylococci, streptococci, pneumonococci, meningococci and gonococci, klebsiella, proteus, serratia, pseudomonas, legionella, diphtheria, salmonella, bacilli, cholera, tetanus, botulism, anthrax, plague, leptospirosis, and Lyme disease bacteria

Some examples of pathogenic fungi causing infections treatable by methods described herein include Candida (albicans, krusei, glabrata, tropicalis, etc.), Cryptococcus neoformans, Aspergillus (fumigatus, niger, etc.), Genus Mucorales (mucor, absidia, rhizopus), Sporothrix schenkii, Blastomyces dermatitidis, Paracoccidioides brasiliensis, Coccidioides immitis and Histoplasma capsulatum.

Some examples of pathogenic parasites causing infections treatable by methods described herein include Entamoeba histolytica, Balantidium coli, Naegleriafowleri, Acanthamoeba sp., Giardia Zambia, Cryptosporidium sp., Pneumocystis carinii, Plasmodium vivax, Babesia microti, Trypanosoma brucei, Trypanosoma cruzi, Leishmania donovani, Toxoplasma gondii, Nippostrongylus brasiliensis.

In all of the above methods, CD40 agonism can be combined with other forms of immunotherapy such as cytokine treatment (e.g., interferons, GM-CSF, G-CSF, IL-2), or bispecific antibody therapy, which provides for enhanced presentation of tumor antigens. See, e.g., Holliger (1993) Proc. Natl. Acad. Sci. (USA) 90:6444-6448; Poljak (1994) Structure 2:1121-1123.

The multispecific antibodies described herein can be used to enhance antigen-specific immune responses by co-administration of the multispecific antibodies described herein with an antigen of interest, e.g., a vaccine. Accordingly, provided herein are methods of enhancing an immune response to an antigen in a subject, comprising administering to the subject: (i) the antigen; and (ii) the multispecific antibodies described herein, such that an immune response to the antigen in the subject is enhanced. The antigen can be, for example, a tumor antigen, a viral antigen, a bacterial antigen or an antigen from a pathogen. Non-limiting examples of such antigens include those discussed in the sections above, such as the tumor antigens (or tumor vaccines) discussed above, or antigens from the viruses, bacteria or other pathogens described above.

As previously described, the multispecific antibodies described herein can be co-administered with one or other more therapeutic agents, e.g., a cytotoxic agent, a radiotoxic agent. The antibody can be linked to the agent (as an immuno-complex) or can be administered separate from the agent. In the latter case (separate administration), the antibody can be administered before, after or concurrently with the agent or can be co-administered with other known therapies, e.g., an anti-cancer therapy, e.g., radiation. Such therapeutic agents include, among others, anti-neoplastic agents such as doxorubicin (adriamycin), cisplatin bleomycin sulfate, carmustine, chlorambucil, dacarbazine and cyclophosphamide hydroxyurea which, by themselves, are only effective at levels which are toxic or subtoxic to a patient. Cisplatin is intravenously administered as a 100 mg/ml dose once every four weeks and adriamycin is intravenously administered as a 60-75 mg/ml dose once every 21 days. Co-administration of the multispecific antibodies described herein with chemotherapeutic agents provides two anti-cancer agents which operate via different mechanisms which yield a cytotoxic effect to human tumor cells. Such co-administration can solve problems due to development of resistance to drugs or a change in the antigenicity of the tumor cells that would render them unreactive with the antibody.

The multispecific antibody (also referred to in plural as “multispecific antibodies”) can be provided to the subject per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the multispecific antibody accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (multispecific antibody) effective to prevent, alleviate or ameliorate symptoms of a disorder (e.g., cancer) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Dosage amount and interval may be adjusted individually to provide tissue levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

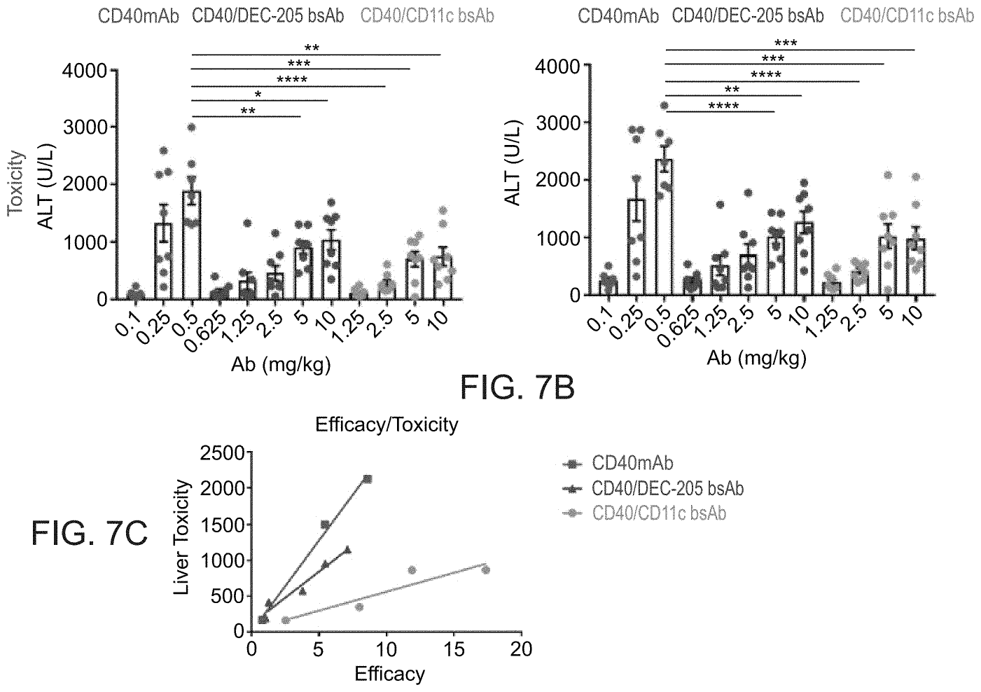

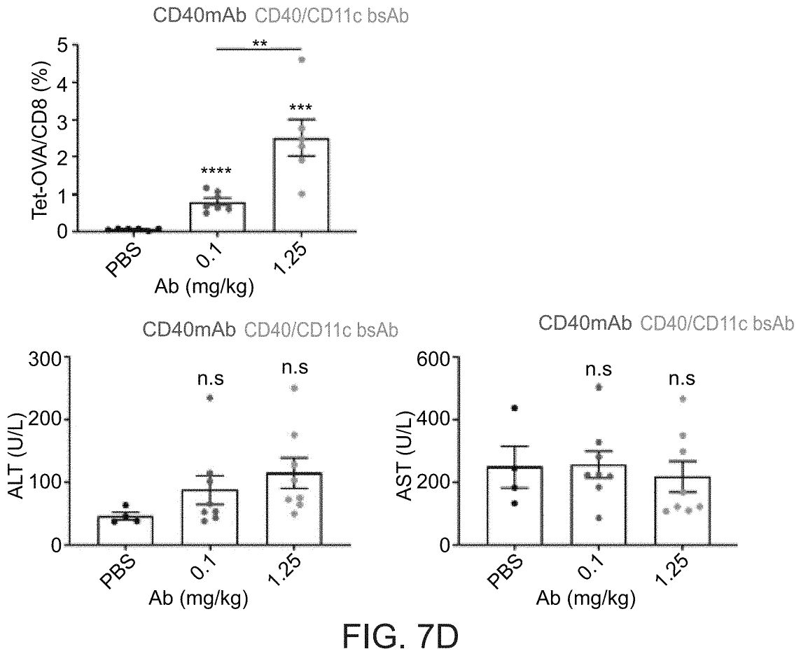

Due to its targeted specificity higher doses can be used of the multispecific antibody than those used with the monospecific CD40 Ab (see FIG. 7A-D).

According to a specific embodiment, the dosing of the multispecific antibody can be 0.1-100 mg/kg.

According to a specific embodiment, the dosing of the multispecific antibody can be 0.1-100 mg/kg. According to a specific embodiment, the dosing of the multispecific antibody can be 0.1-80 mg/kg. According to a specific embodiment, the dosing of the multispecific antibody can be 0.1-60 mg/kg. According to a specific embodiment, the dosing of the multispecific antibody can be 0.1-50 mg/kg. According to a specific embodiment, the dosing of the multispecific antibody can be 0.1-40 mg/kg. According to a specific embodiment, the dosing of the multispecific antibody can be 0.1-30 mg/kg. According to a specific embodiment, the dosing of the multispecific antibody can be 0.1-20 mg/kg. According to a specific embodiment, the dosing of the multispecific antibody can be 0.1-10 mg/kg. According to a specific embodiment, the dosing of the multispecific antibody can be 1-100 mg/kg. According to a specific embodiment, the dosing of the multispecific antibody can be 10-100 mg/kg. According to a specific embodiment, the dosing of the multispecific antibody can be 20-100 mg/kg. According to a specific embodiment, the dosing of the multispecific antibody can be 30-100 mg/kg. According to a specific embodiment, the dosing of the multispecific antibody can be 40-100 mg/kg. According to a specific embodiment, the dosing of the multispecific antibody can be 50-100 mg/kg. According to a specific embodiment, the dosing of the multispecific antibody can be 60-100 mg/kg. According to a specific embodiment, the dosing of the multispecific antibody can be 70-100 mg/kg.

According to a specific embodiment, the dosing of the multispecific antibody can be 1-20 mg/kg. According to a specific embodiment, the dosing of the multispecific antibody can be 1-15 mg/kg. According to a specific embodiment, the dosing of the multispecific antibody can be 1-10 mg/kg. According to a specific embodiment, the dosing of the multispecific antibody can be 1-5 mg/kg. According to a specific embodiment, the dosing of the multispecific antibody can be 2-20 mg/kg. According to a specific embodiment, the dosing of the multispecific antibody can be 4-20 mg/kg. According to a specific embodiment, the dosing of the multispecific antibody can be 6-20 mg/kg. According to a specific embodiment, the dosing of the multispecific antibody can be 8-20 mg/kg. According to a specific embodiment, the dosing of the multispecific antibody can be 10-20 mg/kg. According to a specific embodiment, the dosing of the multispecific antibody can be 12-20 mg/kg. According to a specific embodiment, the dosing of the multispecific antibody can be 15-20 mg/kg. According to a specific embodiment, the dosing of the multispecific antibody can be 18-20 mg/kg. According to a specific embodiment, the dosing of the multispecific antibody can be 1-5 mg/kg. According to a specific embodiment, the dosing of the multispecific antibody can be 2-10 mg/kg. According to a specific embodiment, the dosing of the multispecific antibody can be 5-10 mg/kg.

According to a specific embodiment the dose of the multispecific is at least 5 times, 10 times, 15 times, 20 times or more than that tolerated by anti CD40 monospecific antibody.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

Drug combinations are also contemplated with the multispecific antibody of the present teachings, such as with immune checkpoint modulators such as anti-CTLA4, anti-CD40, anti-41BB, anti-OX40, anti-PD1 or anti-PDL1. According to a specific embodiment, the immune checkpoint modulator is anti-PD1 or anti-PDL1.

It is expected that during the life of a patent maturing from this application many relevant agonistic CD40 antibodies will be developed and the scope of the term anti CD40 antibody is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

Materials and Methods

Mice

Humanized mice containing human Fc receptors (FcγRanull, hFcγRI+, FcγRIIaR131+, FcγRIIb+, FcγRIIIaF158+, and FcγRIIIb+) and human CD40 were generated and characterized as previously described [10][15]. Mice 8-10 weeks of age were used in all experiments. All mice were maintained at The Weizmann Institute of Science Animal Facility Center.

Generation of Anti-CD40/DC Mono or Bi-Specific Abs Fc Variants

Anti-human CD40 antibody 2141 is clone 21.4.1 mentioned in U.S. Pat. No. 7,338,660 (ATCC accession number PTA-3605, native Fc) The variable heavy and light regions of 2141 were synthesized (Genewiz). The parental mouse IgG1 anti-human/mouse DEC-205 antibody, clone HD-109 and the parental Armenian Hamster IgG anti-mouse CD11c antibody, clone N418 producing hybridomas were provided by Rockeffeler University and ATCC, respectively. The variable heavy and light regions sequence of HD-109 and N418 were sequenced from the hybridomas RNA by amplification of cDNA ends method (“anchored” PCR) using RLM-RACE (ThermoFisher) according to the manufacturer's instructions. For amplification of the variable heavy and light regions, PCR was performed using Outer and Inner oligonucleotide: Outer mouse IgG1 heavy chain 5′-TCATTTACCAGGAGAGTGG (SEQ ID NO: 43) and Inner mouse IgG1 heavy chain 5′-AGAGGCTCTTCTCAGTATGGTGGTTGTGC (SEQ ID NO: 44) for clone HD-109 heavy chain, Outer hamster IgG heavy chain 5′-GCTCACGTCCACCACCACACATGT (SEQ ID NO: 45) and Inner hamster IgG heavy chain 5′-GAAATAGCCCTTGACCAGGCATCC (SEQ ID NO: 46) for clone N418 heavy chain, Outer mouse IgG1 kappa light chain 5′-AACACTCATTCCTGTTGAAG (SEQ ID NO: 47) and Inner mouse IgG1 kappa light chain 5′-GCTCTTGACAATGGGTGAAGTTGATGTC (SEQ ID NO: 48) for clone HD-109 light chain, Outer hamster IgG light chain 5′-CTAACACTCATTCCTGTTCAGGGTCTTG (SEQ ID NO: 49) and Inner hamster IgG light chain 5′-GCTGCTCAGGCTGTAGGTGCTGTC (SEQ ID NO: 50) for clone N418 light chain. The variable region sequences of the parental Ab were cloned from the hybridomas and inserted into mammalian expression vectors with mono human IgG1 or human kappa Fc backbones or to bi-specific vectors previously described (Merchant et al., 1998; Ridgway et al., 1996; Schaefer et al., 2011). For correct heavy-light chain pairing, one of the parental mAbs was expressed in the CrossMab format (CH1-CL swapping), while for the other mAb, the wild-type domain architecture was maintained (Schaefer et al., 2011). For heavy chain heterodimerization, point mutations were introduced in the CH3 domain:Y349C/T366S/L368A/Y407V for the 1st mAb (anti DC marker abs); S354C/T366W for the 2nd mAb (anti-CD40) (Merchant et al., 1998; Ridgway et al., 1996). For the generation of Fc-domain variants of human IgG1 (N297A, G237D/P238D/H268D/P271G/A330R(V11) N297 (which does not recruit the receptor), site-directed mutagenesis using specific primers was performed based on the site-directed mutagenesis by PCR (Agilent Technologies) according to the manufacturer's instructions. Mutated plasmid sequences were validated by direct sequencing (Life science core facility, Weizmann Institute of Science). To produce antibodies, antibody heavy and light chain expression vectors were transfected transiently into Expi293 cells (ThermoFisher). The secreted antibodies in the supernatant were purified by protein G Sepharose 4 Fast Flow (GE Healthcare). Purified antibodies were dialyzed in PBS and sterile filtered (0.22 μm). Purity was assessed by SDS-PAGE and Coomassie staining and was estimated to be >90%. Size exclusion chromatography (SEC) was performed using a Superose 6 Increase 10/300GL column (GE Healthcare) on an Äkta Pure 25 FPLC system.

CD40, DEC-205 and CD11c Binding ELISA

Binding specificity and affinity of mono and bi-specific Abs were determined by ELISA using recombinant human CD40 (SINO BIOLOGICAL), human DEC-205 (Sino Biological) and mouse CD11c (R&D Systems). ELISA plates (Nunc) were coated overnight at 4° C. with recombinant extracellular domain of human CD40 or human DEC-205 (1 μg/mL/well) or mouse CD11c (5 μg/mL/well). All sequential steps were performed at room temperature. After being washed, the plates were blocked for 1 hr with 1×PBS with 2% Bovine serum Albumin and were subsequently incubated for 1 hr with serially diluted IgGs (1:5 consecutive dilutions in 1×PBS with 2% Bovine serum Albumin). For dual binding ELISA assay, plates were incubated for 1 hr with biotinylated human CD40 (Acrobiosystems). After washing, plates were incubated for 1 hr with horseradish peroxidase-conjugated anti-human IgG (Jackson ImmunoResearch) or with horseradish peroxidase-conjugated Streptavidin (Biolegend). Detection was performed using a one component substrate solution (TMB) and reactions stopped with the addition of 0.18 M sulphuric acid. Absorbance at 450 nm was immediately recorded using a SpectraMax Plus spectrophotometer (Molecular Devices), and background absorbance from negative control samples was subtracted.

Flow Cytometry

Single cell suspensions were prepared as described above. For surface staining, cells were plated in U-shaped 96-well plates (ThermoFisher) at a concentration of 0.2−1×106 cells in 100 μl PBS. Cells were first stained with LIVE/DEAD™ Fixable blue dead cell stain (ThermoFisher) followed by two washes with PBS, and then resuspended in 25 μl FACS buffer with human or mouse TruStain Fc block (BioLegened), and incubated for 15 minutes at room temperature. Surface antigens were stained in FACS buffer for 30 minutes on ice. Then the cells were washed twice with FACS buffer, resuspended in 150 μl FACS buffer, and analyzed by flow cytometry. For intracellular IL-6 staining, an additional staining step was performed using True-Nuclear™ transcription factor buffer Set Kit (BioLegened) and anti-IL-6 (MP5-20F3) (BioLegened) according to the manufacturer's instructions. All samples were analyzed on CytoFLEX LX (Beckman Coulter). Unless otherwise specified, cell populations were defined by the following markers (BioLegened): DCs: CD45+ (30F11), CD11c+ (N418), MHC II+ (M5/11415.2), F4/80− (BM8). Macrophages: CD45+, CD11b+ (M1/70), MHC II+, F4/80+, Ly6C− (HK1.4), Ly6G− (1A8). Monocytes: CD45+, CD11b+, Ly6C+, F4/80−, CD11c−. B cells: CD45+, CD19+ (1D3). cDC1s: CD45+, MHC II+, CD11c+, XCR1+ (ZET), CD19−, CD64− (10.1), F4/80−, SIRPα− (P84). cDC2s: CD45+, MHC II+, CD11c+, SIRPα+, CD19−, CD64−, F4/80−, XCR1−. Liver cDC1, cDC2, Kupffer cell and non-Kupffer cell macrophages were gated as previously described (Sierro et al., 2017) using the following surface markers: CD45, MHC II, CD11b, CD11c, CD64 (X54-5/7.1), F4/80, Ly6C, Tim4 (RMT4-54), CX3CR1 (SA011F11). For CD40, CD86, CD80 and DEC-205 expression, the following clones were used: CD40 (3/23), CD86 (GL-1), CD80 (16-10A1) and DEC-205 (NLDC-145).

DC Preferential Binding Assay

Spleens were harvested from humanized CD40/FcγR mice, and single cell suspensions were prepared, as described above. Splenocytes were stained using the surface CD19 or CD11b markers, and with CD40 mAb or CD40/CD11c bsAb. Cells were washed twice with FACS buffer and stained with PE-conjugated anti-human IgG (Jackson ImmunoResearch) in FACS buffer on ice, before analysis by flow cytometry.

Human DCs Activation Assay

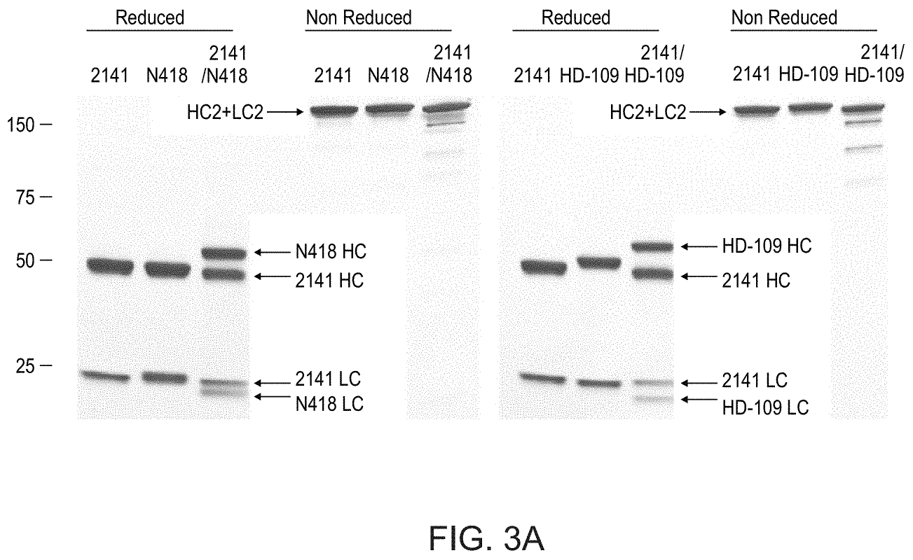

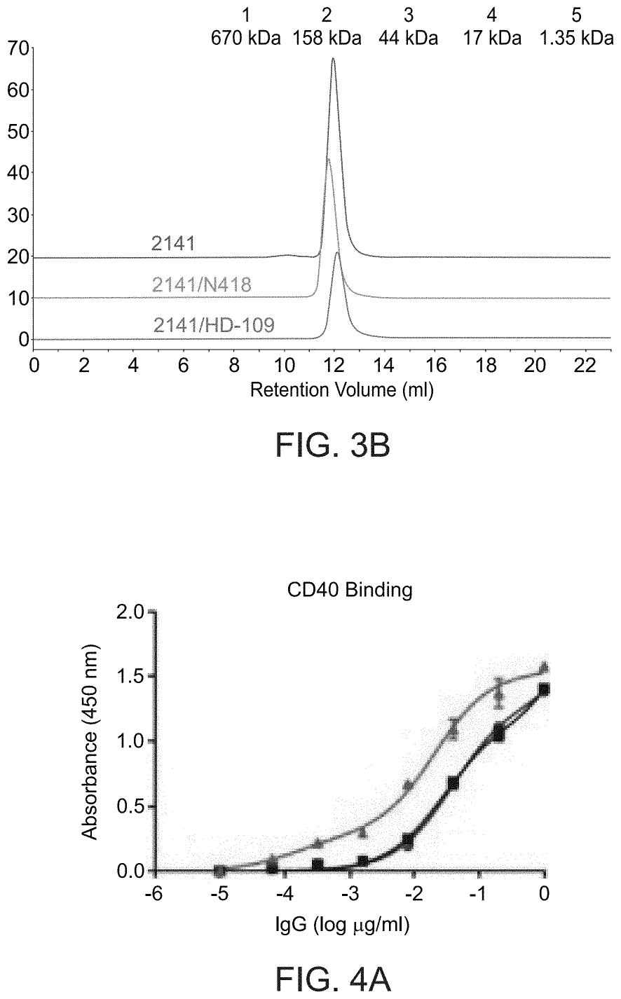

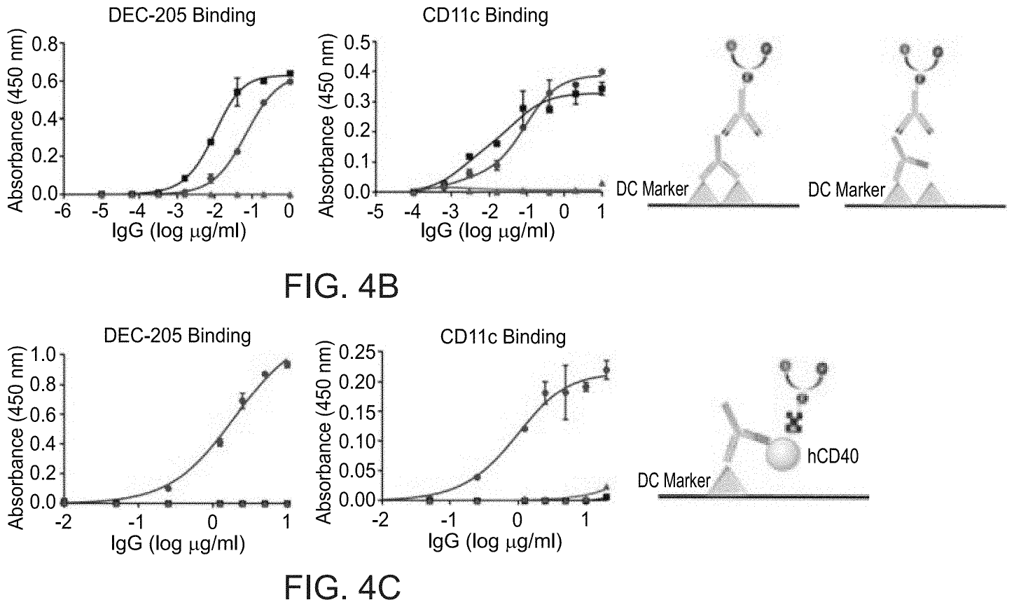

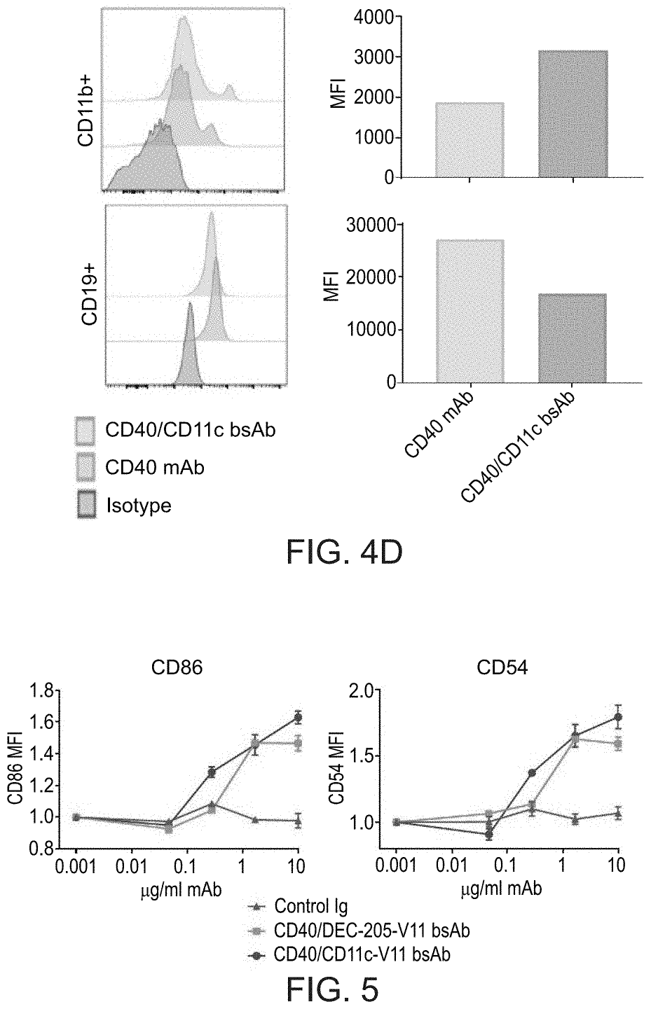

PBMCs were isolated by Ficoll separation (GE Healthcare) of fresh whole blood from healthy donors. Human monocytes (CD14⁺) were isolated using CD14-microbead positive selection according to the manufacturer's instructions (Miltenyi Biotec). Monocytes were cultured 4×10⁶ cells per well in a 6-well plate in RPMI media with 10% heat-inactivated FBS, 1% Pen Strep, 100 ng/mL GM-CSF (Peprotech) and 100 ng/mL IL-4 (Peprotech). Medium was replenished on day 2 and day 5. Monocyte-derived immature DCs were harvested on day 7. For up-regulation analysis of CD54 and CD86, monocyte-derived immature DCs were plated at 1×10⁵ cells/well in U-shape 96-well tissue culture plates (ThermoFisher). Antibodies as indicated in FIGS. 3A and 4B were added to the wells, and incubated overnight at 37° C. Cells were harvested and stained with the following markers: CD86 (BU63), CD54 (HA58). Samples were analyzed by flow cytometry.

OVA-Specific T Cell Response

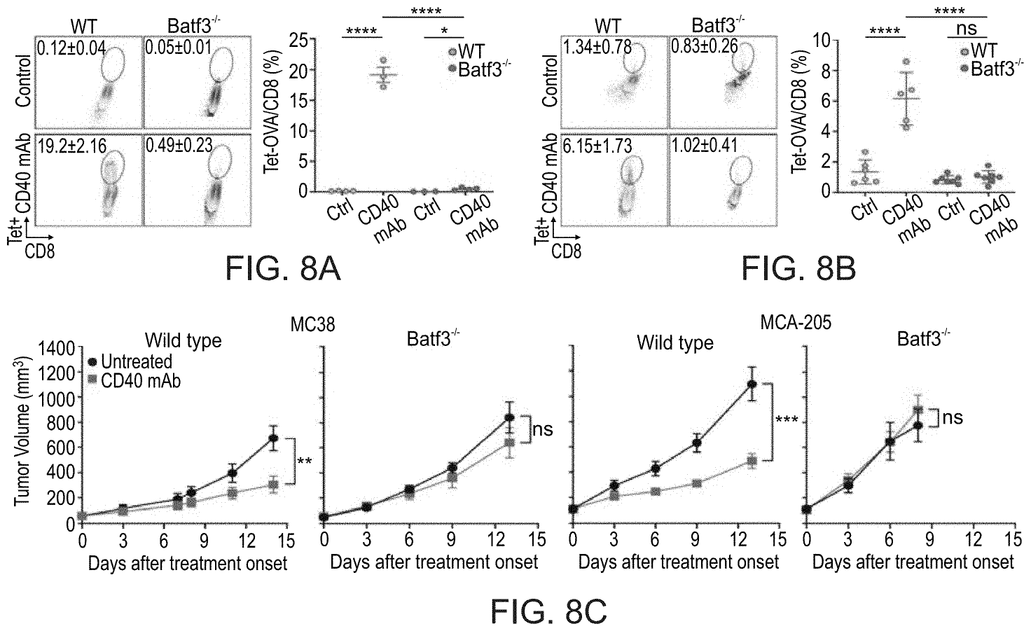

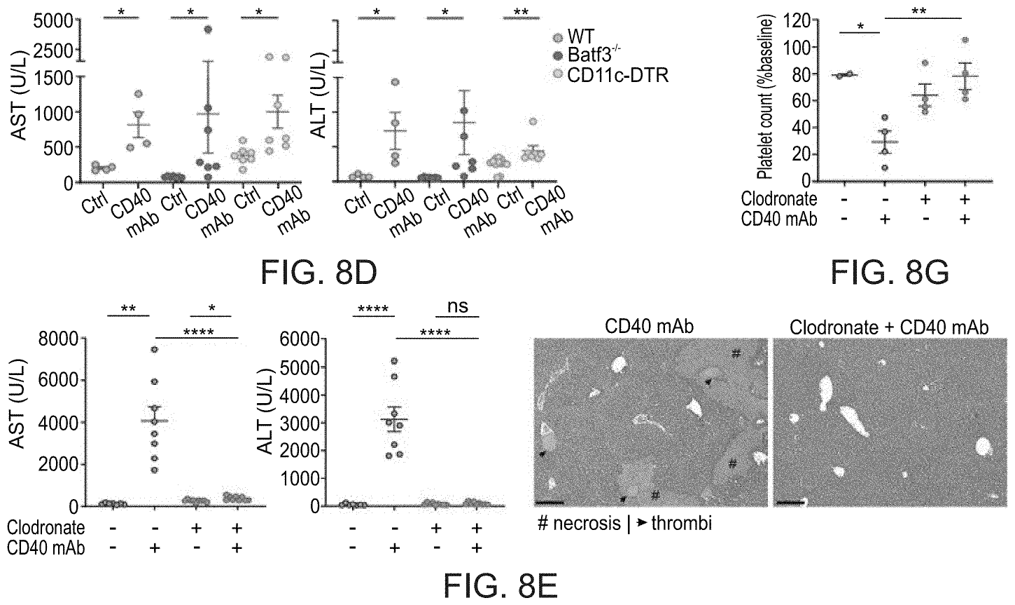

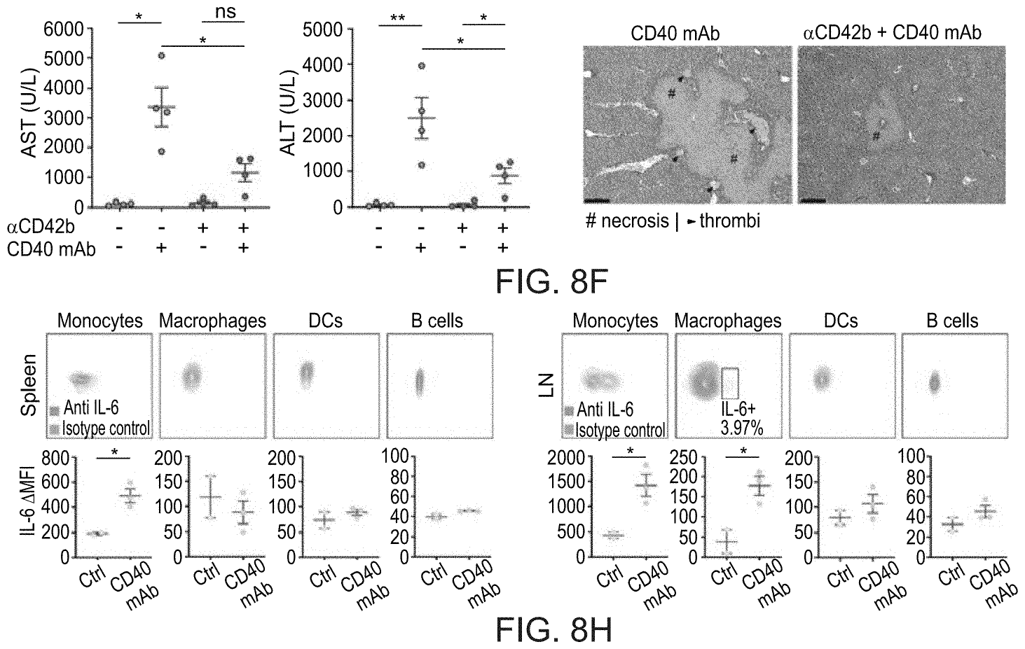

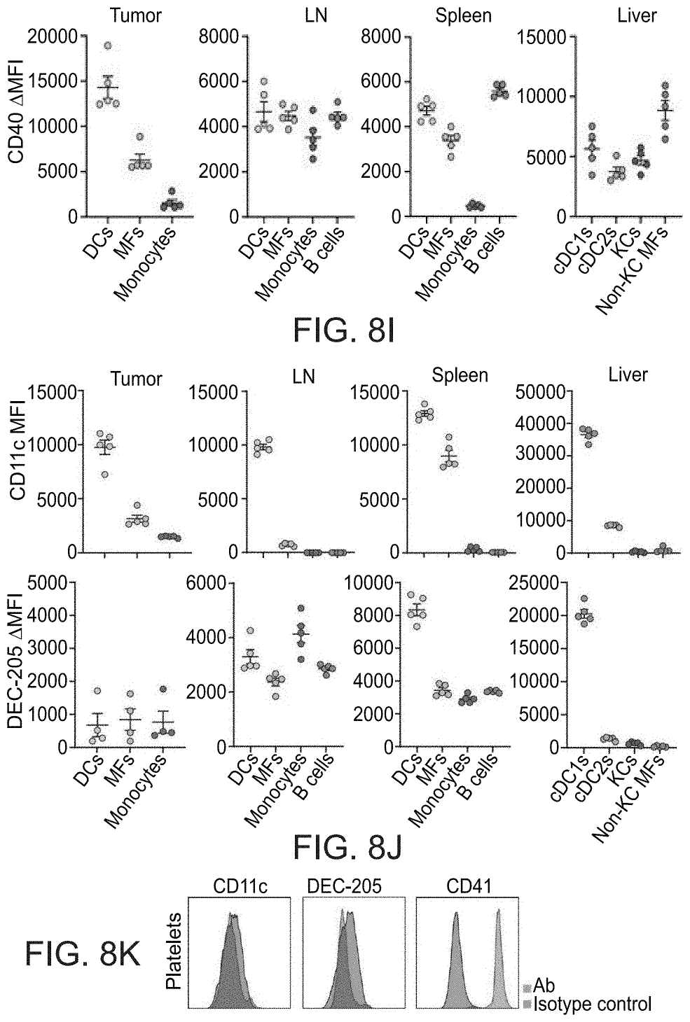

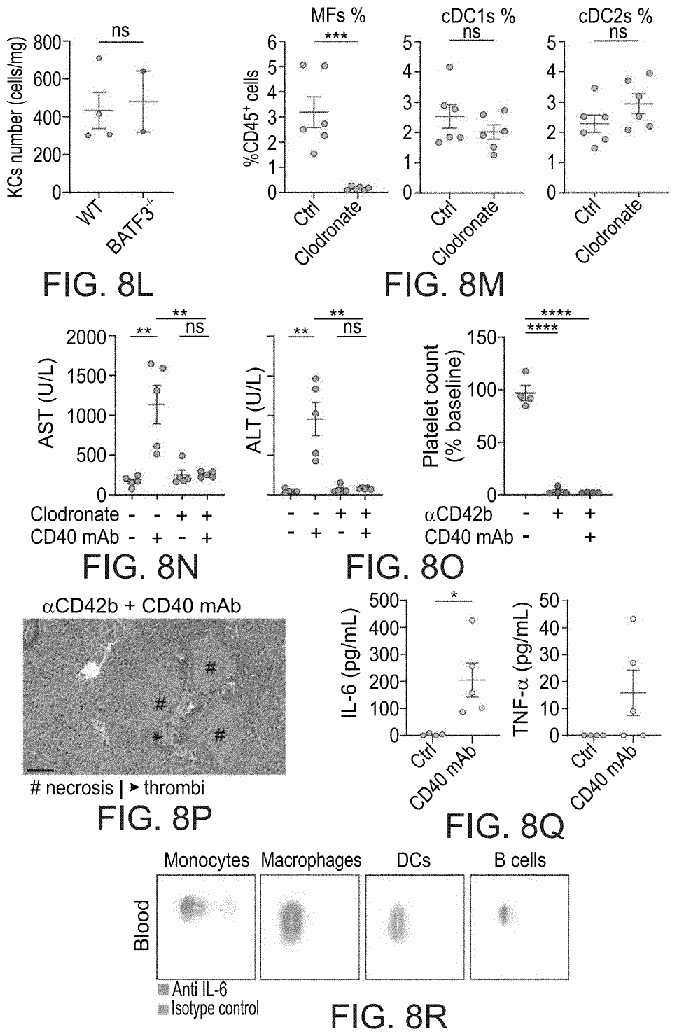

Mice (WT or BATF3^{-/-} in FIG. 8A+B, hCD40/FcγR in all the others) were immunized through intraperitoneal injection of 50 mg/kg of Ovalbumin (Sigma) in the presence or absence of 5 mg/kg of rat anti-mouse CD40 mAb (FGK4.5) (BioXCell) or with 0.1-10 mg/kg of anti-human CD40 mono or bi-specific Abs. After 7 days, peripheral blood was collected and stained with, APC-conjugated anti-CD8α (53-6.7) (BioLegened), and PE-conjugated OVA peptide SIINFEKL H-2b tetramer (Tet-OVA, MBL International Corporation) and analyzed by flow cytometry. For specific T cell response in the OVA-expressing B16 tumor model (B16-OVA), mice were implanted subcutaneously with 2×10⁵ B16-OVA cells. When tumors were established (sum of tumor length and width reached approximately 50 mm³) mice were treated with 5 mg/kg rat anti-mouse CD40 mAb, and treatment was repeated 3 days later. On day 7, peripheral blood was collected and processed as described above.

Transaminases in Serum and H&E Staining

Mice (WT, BATF3^{-/-}, CD11c-DTR or hCD40/FcγR) were treated by intraperitoneal injection with 5 mg/kg of rat anti-mouse CD40 mAb or with 0.1-10 mg/kg of anti-human CD40 mono or bi-specific Abs. After 24 hours, peripheral blood was collected into clot activator serum tubes (Becton Dickinson). Blood was allowed to clot at room temperature for 30 minutes and then centrifuged at 3500 rpm for 10 minutes, and liver transaminase (ALT/AST) levels were determined in the serum by a commercial lab (American Medical Laboratories (AML), Israel). Livers from treated animals were harvested and placed in 4% paraformaldehyde (PFA) overnight, and then paraffin processed and stained with hematoxylin and eosin (H&E) at the Histology & Pathology unit of the Weizmann Institute of Science. Slides were scanned using a Pannoramic scan II scanner (3DHISTECH), and images were obtained with CaseViewer software.

Serum and Intracellular Cytokine Analysis

hCD40/FcγR mice were administered 0.1-10 mg/kg of anti-human CD40 mono or bi-specific Abs by intraperitoneal injection and bled 3 hours later to collect serum. IL-6 and TNF-α levels were quantified using ELISA MAX™ Deluxe Set kits according to the manufacturer's instructions (BioLegend). For intracellular IL-6 detection, mice were administered 2.5 mg/kg or 0.5 mg/kg of anti-human CD40 mAb, and bled 2.5 hours later for intracellular IL-6 staining. Samples were evaluated by flow cytometry as described above.

Tumor Challenge and Treatment

Tumor cell lines were maintained in a humidified incubator at 37o C and 5% CO2, and cultured in complete RPMI medium containing 25 mM HEPES, 1% L-Glutamine, 10% FBS, 1% Pen Strep, 1% Non-Essential Amino acids, and 1% Pyruvate. MC38 (2×106), B16-F10 (4×105), B16-OVA (2×105), and MCA-205 (5×105), were implanted subcutaneously on the right flank of mice, and tumor volumes were blindly measured every 2-3 days with an electronic caliper. Volume is reported using the formula (L22*L1)/2, where L1 is the longest diameter and L2 is the shortest diameter. Seven to 10 days after tumor inoculation, when the sum of tumor length and width reached approximately 50 mm3, mice were randomized by tumor size (day 0), and received treatment by intraperitoneal injection as described for each experiment. WT and BATF3−/− were treated with 100 μg rat anti-mouse CD40 mAbs or control PBS at days 0, 3 and 6. hCD40/FcγR mice were treated with CD40 mAb or CD40/CD11c bsAb at their respective MTDs (0.175 mg/kg and 2.5 mg/kg respectively) on days 0, 2, 4, and 6 and/or with 10 mg/kg PD-1 IgG1-N297A mAb (clone RMP-1-14) at days 0, 3 and 6, or control PBS. Mice were monitored for 8-20 days after treatment initiation, or until the majority of the untreated control group had to be sacrificed due to the Weizmann Institute of Science IACUC limitation for tumor size.

Platelet Count

Platelet count analysis was performed on mouse peripheral blood collected into K2E EDTA tubes (Becton Dickinson). Samples were analyzed using the Sysmex XP-300™ Automated Hematology Analyzer (Sysmex).

Cell-Depletion Studies

Mice were injected intravenously through lateral tail veins 24 hours prior to CD40 treatment with 10 μl/g body weight of Clodronate liposomes (or control PBS liposomes) (Liposoma BV) for macrophage depletion, or with 2 μg/g body weight of anti-CD42b Abs (R300) (EMFRET Analytics) for platelet depletion. To deplete CD11c⁺ DCs from CD11c−DTR mice, diphtheria toxin (DT) (Sigma) was injected intraperitoneally at a dose of 4 μg/kg, 4 and 2 days before CD40 treatment. The depletion efficiency was assessed either by flow cytometry analysis or by blood platelet count.

### Example 2

Results

Conventional type 1 dendritic cells (cDC1) are specialized for priming tumor-specific CD8⁺ T cells, and their frequency and functional state in tumors is associated with enhanced survival of cancer patients and response to checkpoint blockade. Since the CD40 pathway plays an important role in T cell priming by cDC1, and cDC1 are suggested to be the primary target for CD40 mAbs, the present inventors used cDC1-deficient Batf3^{-/-} mice to evaluate the role of cDC1 in different in-vivo activities of CD40 agonistic mAbs. Immunization of wild type mice with ovalbumin (OVA) together with CD40 mAb results in potent activation and systemic expansion of CD8⁺ T cells specific to a peptide derived from the OVA antigen. Expansion of OVA-specific T cells in Batf3^{-/-} mice was significantly impaired, indicating the primary role of cDC1 in the adjuvant in-vivo T-cell priming activity of CD40 agonistic mAbs (FIG. 8A). To model the response to tumor-antigens, mice were inoculated with B16 melanoma cells expressing OVA, and treated them with CD40 mAb (FIG. 8B). When tested 7 days after treatment onset, the present inventors observed OVA-specific CD8⁺ T cells in the blood of wild type but not Batf3^{-/-} mice, indicating that cDC1 are required for priming tumor-specific CD8⁺ T cells upon CD40 mAb treatment. DCs within the TME upregulate their CD40 surface expression compared to DCs in peripheral tissues and compared to other cell types in the TME such as macrophages and monocytes (FIG. 8I), further implicating tumor cDC1 as a major target for CD40 mAb treatment. To evaluate the impact of cDC1 on the overall antitumor effect of CD40 mAb treatment, two tumor models that respond well to anti-CD40 monotherapy were utilized. Mice bearing the MC38 colon adenocarcinoma or MCA-205 fibrosarcoma were treated with CD40 mAb and followed for their tumor growth over time. Significant therapeutic reduction in tumor volume was obtained in wild type mice but not in Batf3^{-/-} animals (FIG. 8C). Thus, cDC1 was identified as an essential cell population required for mediating the tumor-specific CD8+ T cell priming and overall antitumor effect of CD40-targeted immunotherapy.

The present inventors then evaluated the role of DCs in the hepatotoxicity associated with CD40 mAb treatment. Liver damage after CD40 mAb injection was detected by significant elevation of the blood concentration of liver transaminases in wild type, cDC1 deficient Batf3^{-/-}, and even in mice depleted of CD11c⁺ cells prior to CD40 mAb injection (pan-DC deficient CD11c-DTR mice) (FIG. 8D). Thus, DCs are not the cell population that promotes liver toxicity associated with CD40 mAb therapy. It is therefore likely that other CD40 expressing liver-resident cell populations mediate the CD40 mAb-induced hepatotoxicity. Since Kupffer cells and non-Kupffer liver macrophages do not express CD11c (FIG. 8J) and are not depleted in CD11c-DTR mice, and Kupffer cells are not depleted in Batf3^{-/-} mice (FIG. 8L), they are present in all these mouse models and could be responsible for to the observed toxicity. It was therefore decided to further explore the role of these macrophages in liver toxicity.

To identify the CD40⁺ cell populations that mediate the toxicities associated with CD40 immunotherapy, a mouse model was utilized that is fully humanized for CD40 and all Fcγ Receptors (hCD40/FcγRs); this strain recapitulates the dose-limiting toxicities and additional biological activities of human CD40 mAbs reported in clinical settings. To evaluate CD40-mediated hepatotoxicity, hCD40/FcγR mice were injected with a fully human Fc-engineered CD40 agonist (2141-V11), a second-generation Fc-optimized version of Selicrelumab in which the human IgG1 Fc was mutated to selectively enhance binding to the human inhibitory FcγR, FcγRIIB, thereby providing the crosslinking required for optimal CD40 agonism. This molecule is currently being evaluated in early phase clinical trials (ClinicalTrials(dot)gov Identification: NCT04059588, NCT04547777). A single CD40 mAb injection led to rapid elevation in serum levels of liver transaminases, as has been previously reported, indicating liver damage characterized by hepatocyte coagulative necrosis, and sinusoidal thrombosis (FIG. 8E). When systemic depletion of macrophages and phagocytic cells, including liver macrophages and Kupffer cells, but not DCs (FIG. 8M), was induced using clodronate liposomes prior to CD40 mAb injection, liver toxicity was abrogated (FIG. 8E and FIG. 8N). The present inventors hypothesized that this central role of macrophages in mediating the hepatotoxicity can be caused either by FcγRIIB expression by Kupffer cells, leading to local crosslinking of the CD40 mAb and subsequent platelet activation in the liver sinusoids, and/or by direct CD40 pathway activation of liver macrophages.

To distinguish between these possibilities, anti-CD42b mAb to clear circulating platelets (FIG. 8O) was used before CD40 mAb injection. Significant reduction in liver toxicity was observed in the absence of platelets (FIG. 8F). Although AST and ALT levels were somewhat elevated in these settings, their levels were significantly lower compared to CD40 mAb injection without platelet clearance. Histopathological analysis of the livers in these mice revealed only rare and relatively minor events of parenchymal necrosis in the platelet-depleted mice, correlating with their significantly reduced AST and ALT levels, and very few and small fibrin thrombus foci, presumably a result of residual platelets (˜2%) that remain in the circulation after anti-CD42b injection (FIG. 8O). These foci were often found in vesicles near or surrounded by the necrotic parenchyma (FIG. 8P), suggesting a causal relationship between the thrombi and necrosis. Our data thereby support a significant role for platelets in CD40 mAb-induced hepatotoxicity and correlate platelet frequency with the intensity of liver damage. These results support a model in which Kupffer cells and liver macrophages, through their expression of FcγRIIB, mediate platelet crosslinking of CD40 by the anti-CD40 mAb.

Next, the present inventors wished to evaluate the role of macrophages in anti-CD40 mediated thrombocytopenia. A massive reduction in platelet count was observed 24 hr after CD40 mAb injection; this phenotype was abrogated when macrophages were depleted before CD40 mAb injection (FIG. 8G). These results indicate a role for macrophages in mediating anti-CD40 associated thrombocytopenia, and suggest a causal relationship between hepatotoxicity and thrombocytopenia, both induced by platelets and macrophages.

Finally, we wished to evaluate the identity of cells that contribute to the CRS, the third type of CD40 mAb-induced toxicity observed in the clinic. Single injections of CD40 2141-V11 mAb resulted in rapid elevation of serum interleukin-6 (IL-6) and tumor necrosis factor alpha (TNF-α) (FIG. 8Q), similar to that reported for Selicrelumab and other CD40 mAbs in clinical trials. Intracellular IL-6 staining identified monocytes in the blood, lymph nodes (LN) and spleen, together with a rare population of LN macrophages as the cells that rapidly upregulate their IL-6 expression following CD40 mAb injection (FIG. 8H, 8R), suggesting a major role for monocytes in mediating CD40 mAb induced CRS. Collectively, these experiments identify the major cell populations contributing the dose-limiting toxicities associated with CD40 agonists. The present data suggest a primary role of macrophages and platelets in mediating liver toxicity and thrombocytopenia, and of monocytes in mediating IL-6-related CRS.

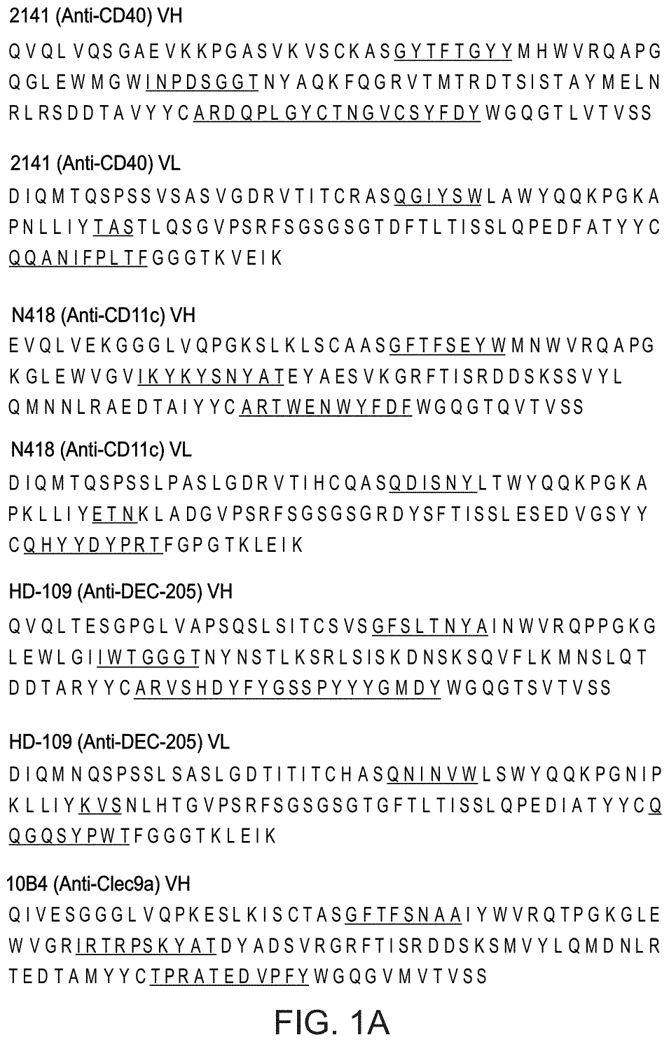

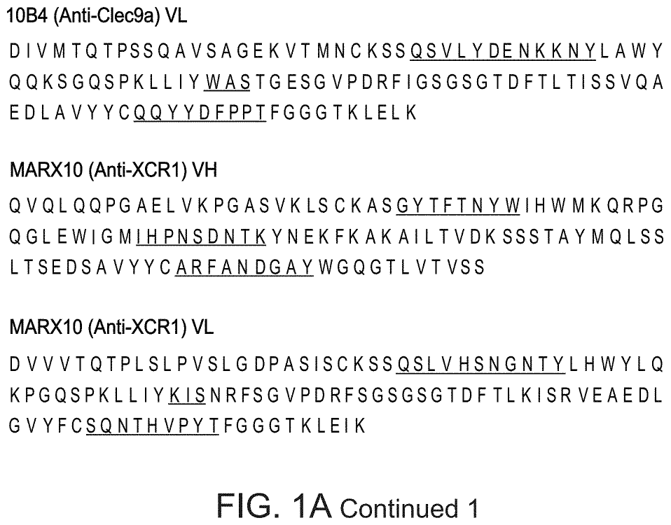

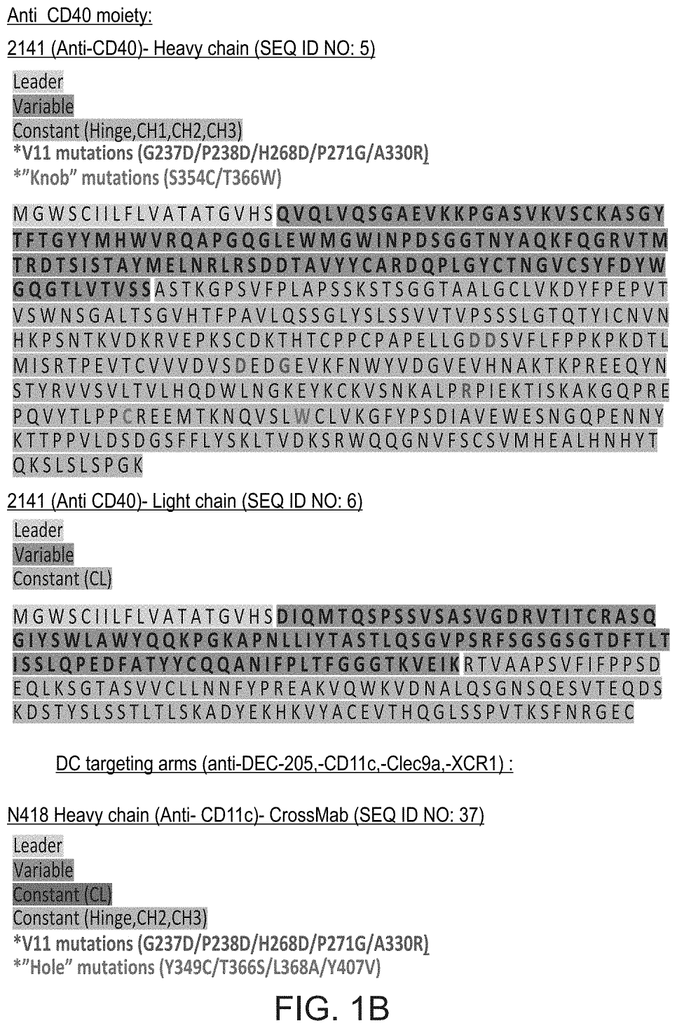

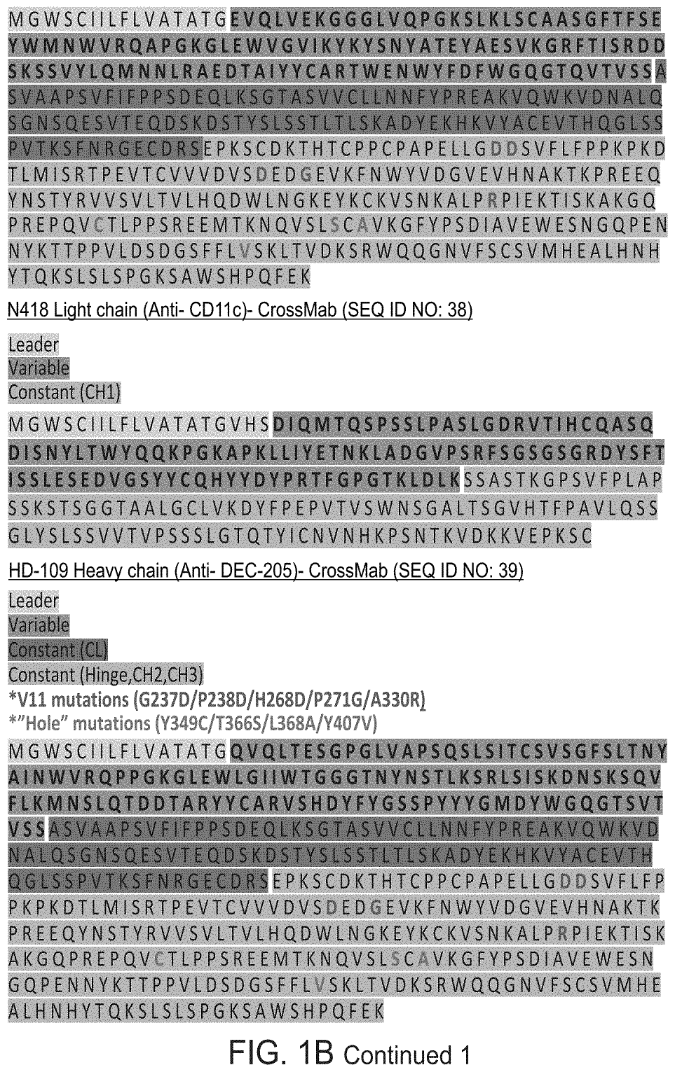

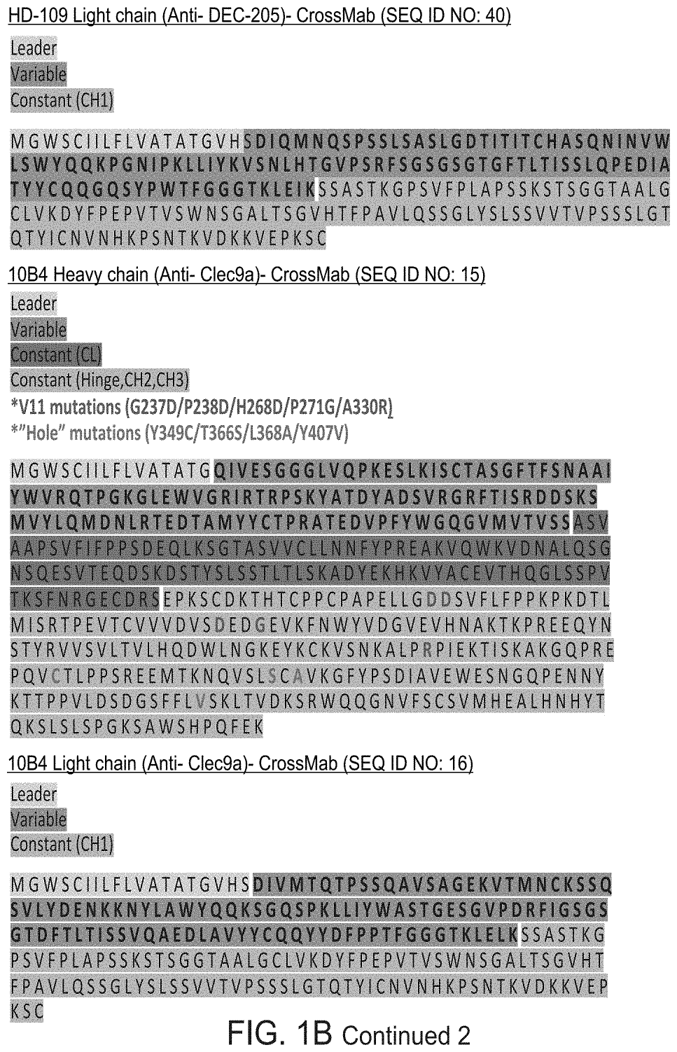

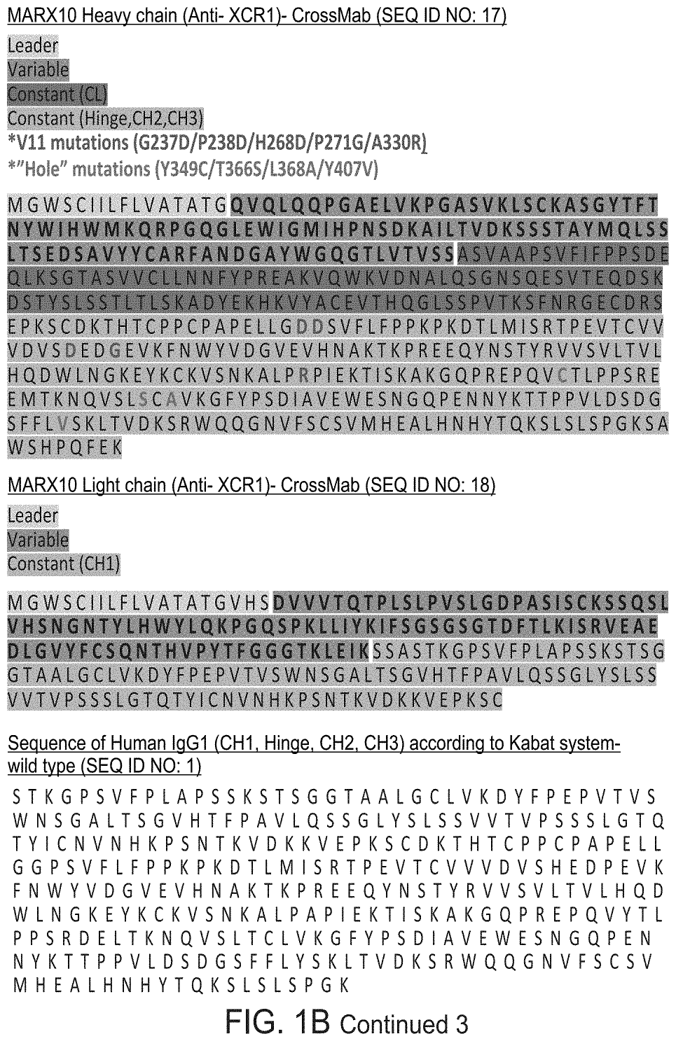

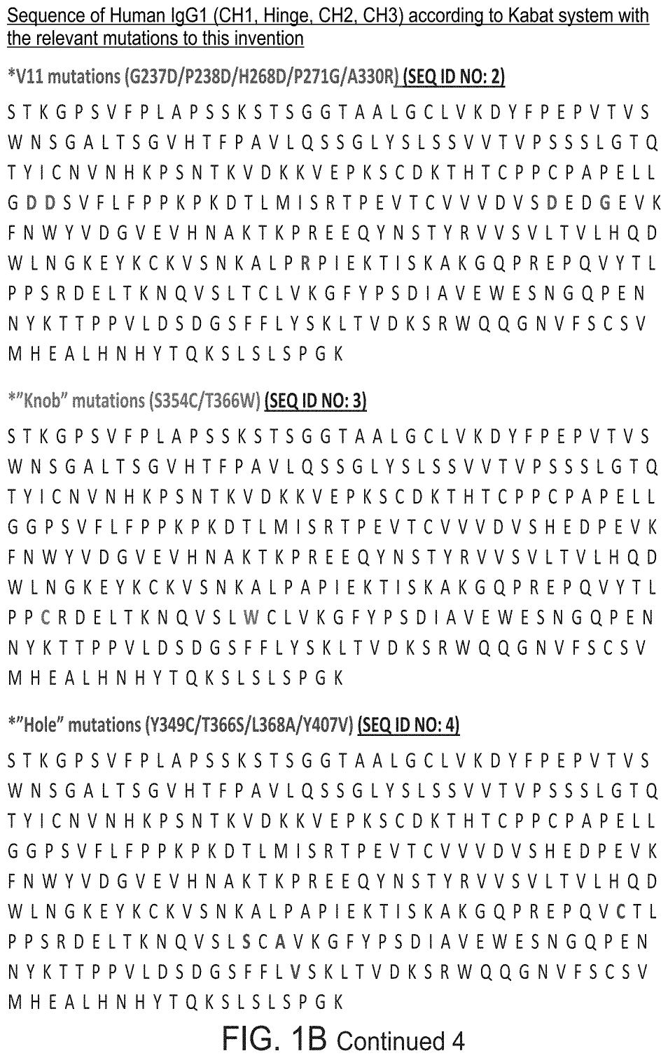

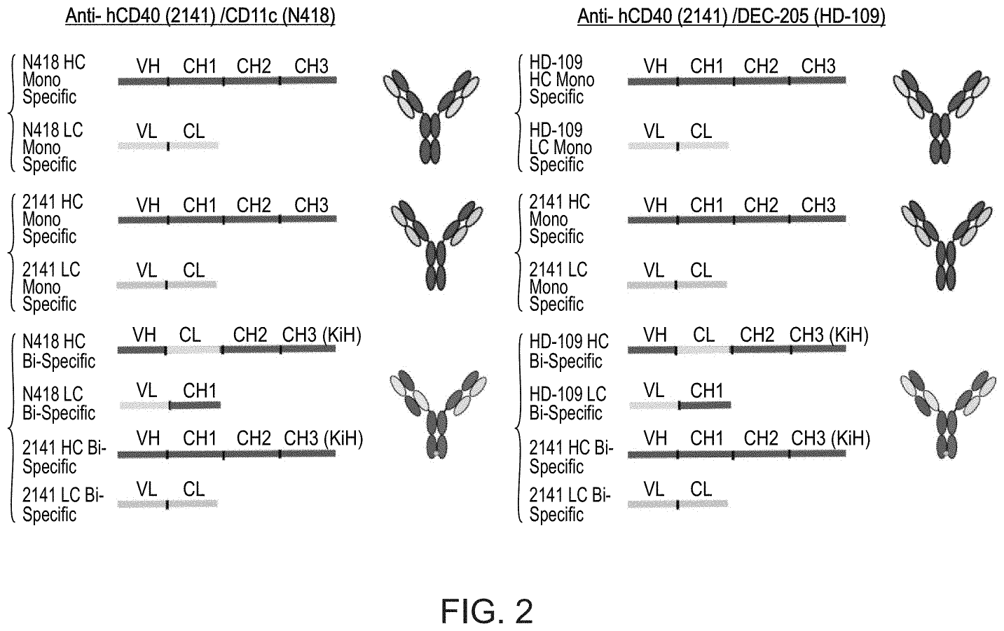

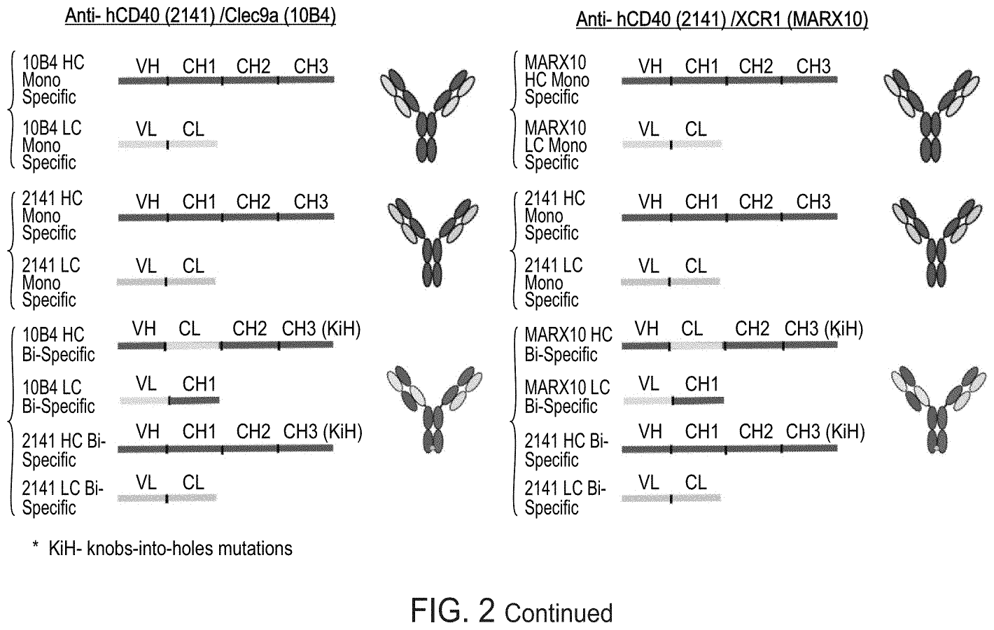

Bispecific antibodies (Abs) targeting CD40 selectively on dendritic cells (DC) were generated by coupling anti-CD40 human Ab 2141 with Abs targeting markers for DCs. In order to clone the desired Abs, the variable regions of the Abs were sequenced from RNA obtained from these hybridoma clones. Once the Abs variable domains sequences were identified (FIG. 1A), they were cloned in frame to the desired IgG constant regions in pre-generated vectors for mono and bispecific heavy and light Ab chains, as described above. DNA constructs were generated containing the translated sequences of anti −CD40 (2141), −CD11c (N418), −DEC-205 (HD109) monospecific, −CD40/CD11c and −CD40/DEC-205 bispecific Abs (FIG. 2). HEK293 cells were transfected with the DNA vector combinations to generate monospecific and bispecific Abs. These five Abs were produced and purified. The formation of the correct heterodimer combination needed to form the CD40/CD11c and CD40/DEC-205 bsAbs was verified by SDS-PAGE and analytical size-exclusion chromatograms (FIG. 3A-B). The simultaneous binding of these bsAbs to both CD40 and CD11c/DEC205 was verified as well as their preferential binding to DC over other cell types negative for DEC-205/CD11c (FIG. 4A-D). Next, the present inventors determined the agonistic activity of these bsAbs and activation of human DCs in vitro (FIG. 5) and mouse T-cells in vivo (FIG. 7A-D). FcγRIIB-requirements for the bsAb activity was determined by comparing the activity of each bsAb while expressed in the context of several Fc scaffolds including WT IgG1, IgG1-V11 (selectively enhanced binding to FcγRIIB), or IgG1-N297A (deglycosylated Fc with no binding to FcγRs). In-vitro and in-vivo experiments showed that these bsAbs required FcγRIIB engagement in order to effectively activate DCs and T-cells (FIG. 6A-B). Specifically, the wild type IgG1s mediated mild activation of DCs, with significantly reduced intensity compared to IgG1-V11 (FIG. 6A). Similarly, the in-vivo T cell activation induced by CD40/CD11c-V11 and CD40/DEC-205-V11 in hCD40/FcγR mice was significantly reduced or completely abrogated by the IgG1 or N297A Fc-silent versions of these bsAbs, respectively (FIG. 6B). Altogether, the present results suggest that the activity of the CD40/DC bsAbs is Fc-dependent, and demonstrate that increased activity is achieved by Fc-engineered bsAbs with enhanced FcγRIIB binding. Therefore, the hIgG1-V11 was selected as the best IgG scaffold for the CD40/DC bsAbs, which was used to further characterize the in vivo properties of Fc-engineered CD40/DC bsAs using this Fc variant. Finally, it was shown that these bsAbs have improved toxicity profiles compared to that of the monospecific 2141 CD40 Ab allowing them to be used in high doses required for optimal antitumor activity. Different doses of bsAbs used in the efficacy experiments described above. Their safety profile was characterized: the bsAbs administered at doses that result with activity, were evaluated for induction of hepatotoxicity. The therapeutic index of each bsAb was compared to that of the parental monomeric 2141-V11 Ab. The maximal safe dose of the optimal CD40/DC bsAb was determined and the present inventors compared the therapeutic efficacy of this safe dose to that of the pre-determined MTD of the monospecific parental 2141-V11 (FIGS. 7A-D). From this study, it can be concluded that the DC-targeted bsAb format of 2141 is able to achieve an increased therapeutic window at least in the matter of liver toxicity.

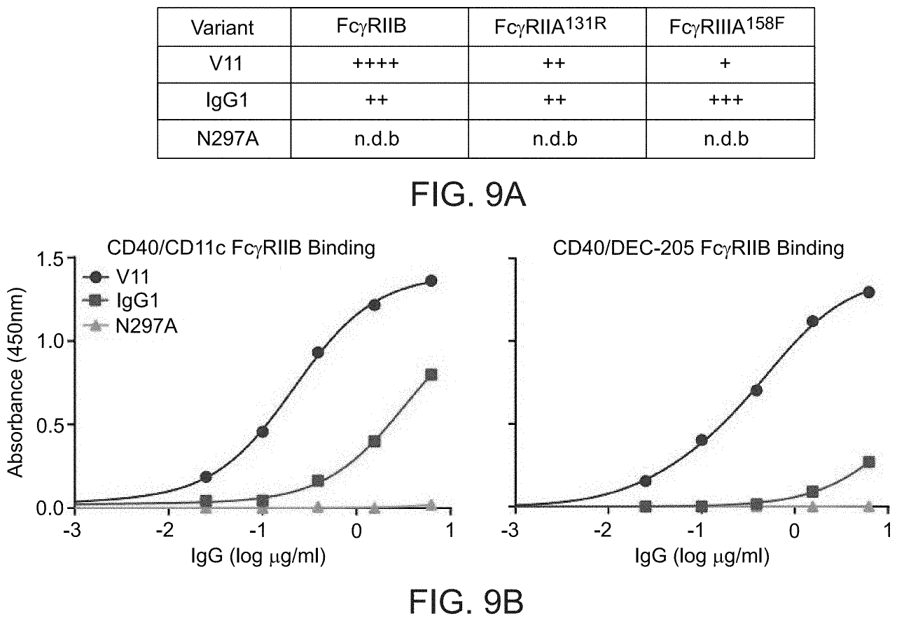

The present inventors generated variants of each bsAb based on three different Fc scaffolds exhibiting distinct binding properties to human FcγRs: wild type hIgG1, hIgG1-N297A (deglycosylated Fc with no binding to FcγRs), and hIgG1-V11 (Fc point mutations that enhance binding to the inhibitory hFcγRIIB) (FIG. 9A-B).

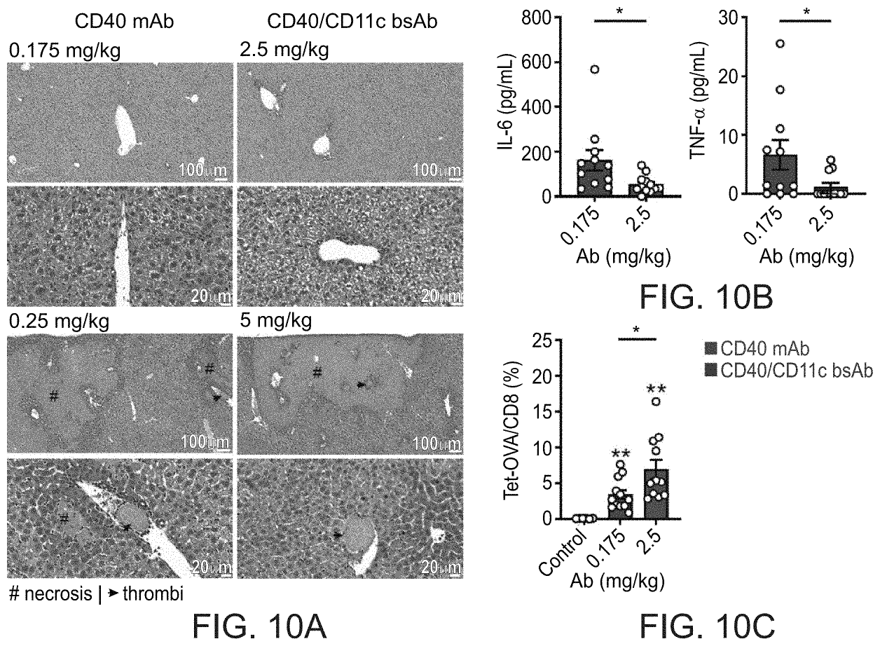

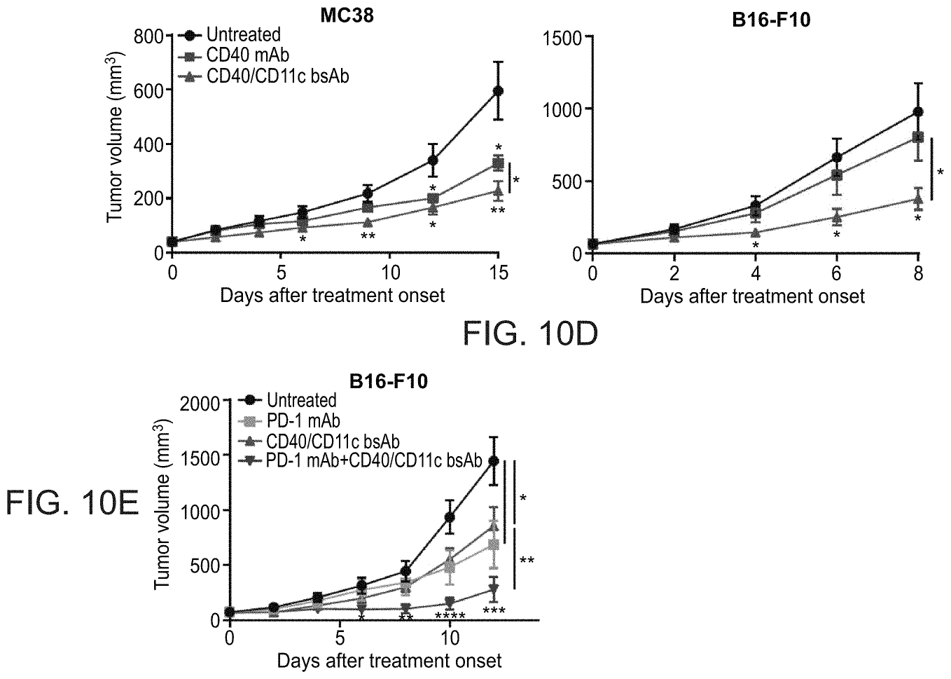

To determine if increased therapeutic efficacy can be mediated by CD40/CD11c bsAb compared to CD40 mAb when they are administered at non-toxic doses, the present inventors determined their MTD based on their degree of hepatotoxicity. They identified 0.175 mg/kg and 2.5 mg/kg for CD40 mAb and CD40/CD11c bsAb, respectively, as the maximal doses that do not cause elevation of serum ALT/AST beyond the standard homeostatic values (FIG. 7B) with no signs of liver necrosis and thrombus (FIG. 10A). At the next higher tested doses both reagents induced significant toxicity, as manifested by elevation in ALT/AST levels, and liver histology revealed extensive hepatocyte necrosis and fibrin thrombi in treated mice. The present inventors evaluated the serum IL-6 and TNF-α levels at these MTDs, and found significant less elevation of these CRS-related cytokines after injection of CD40/CD11c bsAb compared to CD40 mAb (FIG. 10B). At their MTDs, CD40/CD11c bsAb induced significantly enhanced T cell activation compared to the parental CD40 mAb (FIG. 10C), supporting the increased therapeutic index of the bsAb design.

Next, the present inventors wished to determine if this improvement in T cell priming is translated to increased therapeutic anti-tumor activity. To this end, they treated tumor bearing mice with CD40/CD11c bsAb and CD40 mAb at their respective MTDs (FIG. 10D). CD40/CD11c bsAb treatments in both MC38 colon and B16-F10 melanoma tumor models, resulted in significantly improved control of tumor growth compared to CD40 mAb treatment. No signs of liver toxicity were detected in the treated mice at the end of the treatment course, verifying the safety profile of these treatment regimens. These results suggest improved antitumor potency of the CD40/CD11c bsAb over CD40 mAb when they are dosed at their maximal safe dose.

CD40 agonistic mAbs were shown to synergize with PD-1 blockade in several preclinical models [20][21]; this combination demonstrated clinical activity in metastatic ductal pancreatic adenocarcinoma (PDAC) patients in early phase trials [22], and is being evaluated for additional clinical indications. They therefore evaluated whether the CD40/DC11c bsAb retains such synergistic activity. hFcγR/CD40 mice bearing B16 melanoma tumors were treated with either PD-1 mAb, CD40/CD11c bsAb or their combination (FIG. 10E). Increased antitumor activity was induced by the combination therapy compared to each mono-therapy, supporting the potential of combining CD40/DC bsAb and anti-PD1/L1 for enhanced therapy.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

(other references are cited throughout the application)
[1] P. Sharmea, “The future of immue checkpoint therapy,” Science (80-.)., vol. 348, no. 6230, pp. 56-61, 2014.
[2] M. K. Callahan, M. A. Postow, and J. D. Wolchok, “Targeting T Cell Co-receptors for Cancer Therapy,” Immunity, vol. 44, no. 5, pp. 1069-1078, 2016.
[3] R. H. Vonderheide, “The Immune Revolution: A Case for Priming, Not Checkpoint,” Cancer Cell, vol. 33, no. 4, pp. 563-569, 2018.
[4] A. B. Alexandroff et al., “Role for CD40-CD40 ligand interactions in the immune response to solid tumours,” Mol. Immunol., vol. 37, no. 9, pp. 515-526, 2000.
[5] R. H. Vonderheide and M. J. Glennie, “Agonistic CD40 antibodies and cancer therapy,” Clin. Cancer Res., vol. 19, no. 5, pp. 1035-1043, 2013.
[6] L. P. Richman and R. H. Vonderheide, “Role of Crosslinking for Agonistic CD40 Monoclonal Antibodies as Immune Therapy of Cancer,” Cancer Immunol. Res., vol. 2, no. 1, pp. 19-26, 2014.
[7] A. L. White et al., “Interaction with Fc RIIB Is Critical for the Agonistic Activity of Anti-CD40 Monoclonal Antibody,” J. Immunol., vol. 187, no. 4, pp. 1754-1763, 2011.
[8] R. H. Vonderheide et al., “Phase i study of the CD40 agonist antibody CP-870,893 combined with carboplatin and paclitaxel in patients with advanced solid tumors,” Oncoimmunology, vol. 2, no. 1, pp. 1-10, 2013.
[9] R. H. Vonderheide et al., “Clinical activity and immune modulation in cancer patients treated with CP-870,893, a novel CD40 agonist monoclonal antibody,” J. Clin. Oncol., vol. 25, no. 7, pp. 876-883, 2007.
[10] R. Dahan, B. C. Barnhart, F. Li, A. P. Yamniuk, A. J. Korman, and J. V. Ravetch, “Therapeutic Activity of Agonistic, Human Anti-CD40 Monoclonal Antibodies Requires Selective FcγR Engagement,” Cancer Cell, vol. 29, no. 6, pp. 820-831, 2016.
[11] D. A. Knorr, R. Dahan, and J. V Ravetch, “Toxicity of an Fc-engineered anti-CD40 antibody is abrogated by intratumoral injection and results in durable antitumor immunity [Immunology and Inflammation],” Proc. Natl. Acad. Sci. U.S.A, pp. 2-7, 2018.
[12] Y. Mazor et al., “Improving target cell specificity using a novel monovalent bispecific IgG design,” MAbs, vol. 7, no. 2, pp. 377-389, 2015.
[13] C. G. Park et al., “Generation of anti-human DEC205/CD205 monoclonal antibodies that recognize epitopes conserved in different mammals,” J. Immunol. Methods, vol. 377, no. 1-2, pp. 15-22, 2012.
[14] C. Demangel, J. Zhou, A. B. H. Choo, G. Shoebridge, G. M. Halliday, and W. J. Britton, “Single chain antibody fragments for the selective targeting of antigens to dendritic cells,” Mol. Immunol., vol. 42, no. 8, pp. 979-985, 2005.
[15] I. Caminschi et al., “The dendritic cell subtype-restricted C-type lectin Clec9A is a target for vaccine enhancement,” Blood, vol. 112, no. 8, pp. 3264-3273, 2008.
[16] D. Sancho et al., “Tumor therapy in mice via antigen targeting to a novel, DC-restricted C-type lectin,” J. Clin. Invest., vol. 118, no. 6, pp. 2098-2110, 2008.
[17] A. Bachem et al., “Expression of XCR1 characterizes the Batf3-dependent lineage of dendritic cells capable of antigen cross-presentation,” Front. Immunol., vol. 3, no. JUL, pp. 1-12, 2012.
[18] R. Dahan, B. C. Barnhart, F. Li, A. P. Yamniuk, A. J. Korman, and J. V. Ravetch, “Therapeutic Activity of Agonistic, Human Anti-CD40 Monoclonal Antibodies Requires Selective FcR Engagement,” Cancer Cell, vol. 29, no. 6, pp. 820-831, 2016.
[19] P. Smith, D. J. DiLillo, S. Bournazos, F. Li, and J. V. Ravetch, “Mouse model recapitulating human Fcγ receptor structural and functional diversity,” Proc. Natl. Acad. Sci. U.S.A, vol. 109, no. 16, pp. 6181-6186, 2012.
[20] H. S. Ma et al., “A CD40 Agonist and PD-1 Antagonist Antibody Reprogram the Microenvironment of Nonimmunogenic Tumors to Allow T-cell—Mediated Anticancer Activity,” Cancer Immunol. Res., 2019.
[21] S. F. Ngiow et al., “Agonistic CD40 mAb-driven IL12 reverses resistance to anti-PD1 in a T-cell-rich tumor,” Cancer Res., 2016.
[22] M. H. O. Hara et al., “CD40 agonistic monoclonal antibody APX005M (sotigalimab) and chemotherapy, with or without nivolumab, for the treatment of metastatic pancreatic adenocarcinoma: an open-label, multicentre, phase 1b study,” vol. 22, no. January, 2021.

What is claimed is:

1. A multispecific antibody comprising a first moiety, which binds and activates CD40, a second moiety, which specifically binds a dendritic cell (DC) and a third moiety comprising a modified Fc region of said multispecific antibody for enhancing specificity and affinity of binding to FcγRIIb.

2. The multispecific antibody of claim 1, being a trifunctional antibody.

3. The multispecific antibody of claim 1, wherein said second moiety binds a DC marker selected from the group consisting of CD11c, CD11b, DEC-205, BDCA-1, CD8, CD8α, CD103 and MHC-ClassII (e.g., HLA-DR), CD141, FLT3, CD13, CD1c, Clec9a, PD-L1, and XCR1.

4. The multispecific antibody of claim 2, wherein said second moiety binds CD11c.

5. The multispecific antibody of claim 2, wherein said second moiety binds DEC-205.

6. The multispecific antibody of claim 2, wherein said second moiety binds Clec9a.

7. The multispecific antibody of claim 2, wherein said second moiety binds XCR1.

8. The multispecific antibody of claim 5, comprising a first moiety comprising complementary determining regions as set forth in SEQ ID NOs: 19-21 in a heavy chain with an N to C orientation and complementary determining regions as set forth in SEQ ID NOs: 22-24 in a light chain with an N to C orientation.

9. The multispecific antibody of claim 8, wherein said modified Fc region comprises mutations as in SEQ ID NO: 2.

10. The multispecific antibody of claim 9, comprising knobs-into-holes mutations.

11. The multispecific antibody of claim 10, wherein said mutations are in a CH3 domain of a first antibody of said bispecific antibody comprising Y349C/T366S/L368A/Y407V and in a CH3 domain of a second antibody of said multispecific antibody comprising S354C/T366W.

12. The multispecific antibody of claim 11, comprising SEQ ID NOs: 5 and 6 and either of SEQ ID NOs: 37 and 38; SEQ ID NOs: 39 and 40; SEQ ID NOs: 15 and 16; or SEQ ID NOs: 17 and 18.

13. A pharmaceutical composition comprising the multispecific antibody of claim 11.

14. A method of stimulating an immune response in a subject in need thereof, the method comprising administering to the subject a therapeutically effective amount of the pharmaceutical composition of claim 13, thereby stimulating the immune response in the subject.

15. The method of claim 14, wherein the subject has a tumor and an immune response against the tumor is stimulated.

16. The method of claim 14, wherein the subject has a chronic viral infection and an immune response against the viral infection is stimulated.

17. A method of treating cancer in a subject in need thereof, the method comprising administering to the subject a therapeutically effective amount of the pharmaceutical composition of claim 13, thereby treating cancer in the subject.

18. The method of claim 17, wherein the cancer is selected from the group consisting of: bladder cancer, breast cancer, uterine/cervical cancer, ovarian cancer, prostate cancer, testicular cancer, esophageal cancer, gastrointestinal cancer, pancreatic cancer, colorectal cancer, colon cancer, kidney cancer, head and neck cancer, lung cancer, stomach cancer, germ cell cancer, bone cancer, liver cancer, thyroid cancer, skin cancer, neoplasm of the central nervous system, lymphoma, leukemia, myeloma, sarcoma, and virus-related cancer.

19. A method of treating a chronic viral infection in a subject in need thereof, the method comprising administering to the subject a therapeutically effective amount of the pharmaceutical composition of claim 13, thereby treating the chronic viral infection in the subject.

20. The method of claim 19, further comprising the use or administration of a checkpoint modulator.


SEQUENCE LISTING (63 sequences)

SEQ ID NO: 1 - protein, 329 aa, Homo sapiens
STKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 329 aa, synthetic construct
  REGION 1..329: note = human igg1 v11 amino acid sequence
STKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGDDSVFLFPPKPKDTLMISRTPEVTCVVVDVSDEDGEVKFNWYVDGVEVHNAKTKPREEQYNSTYRVVSVLTVLHQDWLNGKEYKCKVSNKALPRPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 329 aa, synthetic construct
  REGION 1..329: note = Human IgG1 amino acid sequence with Knob mutations (S354C/T366W)
STKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 329 aa, synthetic construct
  REGION 1..329: note = Human IgG1 amino acid sequence with Hole mutations(Y349C/T366S/L368A/Y407V)
STKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 5 - protein, 475 aa, synthetic construct
  REGION 1..475: note = 2141 (Anti-CD40) Heavy chain amino acid sequence
MGWSCIILFLVATATGVHSQVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGDDSVFLFPPKPKDTLMISRTPEVTCVVVDVSDEDGEVKFNWYVDGVEVHNAKTKPREEQYNSTYRVVSVLTVLHQDWLNGKEYKCKVSNKALPRPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 233 aa, synthetic construct
  REGION 1..233: note = 2141 (Anti-CD40) Light chain amino acid sequence
MGWSCIILFLVATATGVHSDIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 120 aa, synthetic construct
  REGION 1..120: note = n418 vh amino acid sequence
EVQLVEKGGGLVQPGKSLKLSCAASGFTFSEYWMNWVRQAPGKGLEWVGVIKYKYSNYATEYAESVKGRFTISRDDSKSSVYLQMNNLRAEDTAIYYCARTWENWYFDFWGQGTQVTVSS

SEQ ID NO: 8 - protein, 231 aa, synthetic construct
  REGION 1..231: note = n418 vl amino acid sequence
MGWSCIILFLVATATGVHSDIQMTQSPSSLPASLGDRVTIHCQASQDISNYLTWYQQKPGKAPKLLIYETNKLADGVPSRFSGSGSGRDYSFTISSLESEDVGSYYCQHYYDYPRTFGPGTKLDLKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 9 - protein, 494 aa, synthetic construct
  REGION 1..494: note = hd109 vh amino acid sequence
MGWSCIILFLVATATGQVQLTESGPGLVAPSQSLSITCSVSGFSLTNYAINWVRQPPGKGLEWLGIIWTGGGTNYNSTLKSRLSISKDNSKSQVFLKMNSLQTDDTARYYCARVSHDYFYGSSPYYYGMDYWGQGTSVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDRSEPKSCDKTHTCPPCPAPELLGDDSVFLFPPKPKDTLMISRTPEVTCVVVDVSDEDGEVKFNWYVDGVEVHNAKTKPREEQYNSTYRVVSVLTVLHQDWLNGKEYKCKVSNKALPRPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKSAWSHPQFEK

SEQ ID NO: 10 - protein, 231 aa, synthetic construct
  REGION 1..231: note = hd109 vl amino acid sequence
MGWSCIILFLVATATGVHSDIQMNQSPSSLSASLGDTITITCHASQNINVWLSWYQQKPGNIPKLLIYKVSNLHTGVPSRFSGSGSGTGFTLTISSLQPEDIATYYCQQGQSYPWTFGGGTKLEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 11 - protein, 126 aa, synthetic construct
  REGION 1..126: note = cp870 vh amino acid sequence
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSS

SEQ ID NO: 12 - protein, 107 aa, synthetic construct
  REGION 1..107: note = cp870 vl amino acid sequence
DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIK

SEQ ID NO: 13 - protein, 112 aa, synthetic construct
  REGION 1..112: note = 19g3 vh amino acid sequence
EVQLVESGGGLVKPGGSLKLSCAASGFTFSSYAMSWVRQSPEKRLEWVAEISGGGSYTYYPDTVTGRFTISRDNAKNTLYLEMSSLRSEDTAMYYCASRAYWGQGTLVTVSS

SEQ ID NO: 14 - protein, 112 aa, synthetic construct
  REGION 1..112: note = 19g3 vl amino acid sequence
DVLMTQTPLSLPVSLGDQASISCRSSQSIVHSNGNTYFEWYLQKPGQSPKLLIYKVSNRFSGVPDRFSGSGSGTDFTLKISRVEAEDLGVYYCFQGSHVPYTFGGGTKLEIK

SEQ ID NO: 15 - protein, 118 aa, synthetic construct
  REGION 1..118: note = 10b4 vh amino acid sequence
QIVESGGGLVQPKESLKISCTASGFTFSNAAIYWVRQTPGKGLEWVGRIRTRPSKYATDYADSVRGRFTISRDDSKSMVYLQMDNLRTEDTAMYYCTPRATEDVPFYWGQGVMVTVSS

SEQ ID NO: 16 - protein, 113 aa, synthetic construct
  REGION 1..113: note = 10b4 vh amino acid sequence
DIVMTQTPSSQAVSAGEKVTMNCKSSQSVLYDENKKNYLAWYQQKSGQSPKLLIYWASTGESGVPDRFIGSGSGTDFTLTISSVQAEDLAVYYCQQYYDFPPTFGGGTKLELK

SEQ ID NO: 17 - protein, 116 aa, synthetic construct
  REGION 1..116: note = mrx10 vh amino acid sequence
QVQLQQPGAELVKPGASVKLSCKASGYTFTNYWIHWMKQRPGQGLEWIGMIHPNSDNTKYNEKFKAKAILTVDKSSSTAYMQLSSLTSEDSAVYYCARFANDGAYWGQGTLVTVSS

SEQ ID NO: 18 - protein, 112 aa, synthetic construct
  REGION 1..112: note = mrx10 vh amino acid sequence
DVVVTQTPLSLPVSLGDPASISCKSSQSLVHSNGNTYLHWYLQKPGQSPKLLIYKISNRFSGVPDRFSGSGSGTDFTLKISRVEAEDLGVYFCSQNTHVPYTFGGGTKLEIK

SEQ ID NO: 19 - protein, 8 aa, synthetic construct
  REGION 1..8: note = Complementarity-determining region (CDR)
GYTFTGYY

SEQ ID NO: 20 - protein, 8 aa, synthetic construct
  REGION 1..8: note = Complementarity-determining region (CDR)
INPDSGGT

SEQ ID NO: 21 - protein, 19 aa, synthetic construct
  REGION 1..19: note = Complementarity-determining region (CDR)
ARDQPLGYCTNGVCSYFDY

SEQ ID NO: 22 - protein, 6 aa, synthetic construct
  REGION 1..6: note = Complementarity-determining region (CDR)
QGIYSW

SEQ ID NO: 23 - skipped

SEQ ID NO: 24 - protein, 10 aa, synthetic construct
  REGION 1..10: note = Complementarity-determining region (CDR)
QQANIFPLTF

SEQ ID NO: 25 - protein, 8 aa, synthetic construct
  REGION 1..8: note = Complementarity-determining region (CDR)
GFTFSEYW

SEQ ID NO: 26 - protein, 10 aa, synthetic construct
  REGION 1..10: note = Complementarity-determining region (CDR)
IKYKYSNYAT

SEQ ID NO: 27 - protein, 11 aa, synthetic construct
  REGION 1..11: note = Complementarity-determining region (CDR)
ARTWENWYFDF

SEQ ID NO: 28 - protein, 6 aa, synthetic construct
  REGION 1..6: note = Complementarity-determining region (CDR)
QDISNY

SEQ ID NO: 29 - skipped

SEQ ID NO: 30 - protein, 9 aa, synthetic construct
  REGION 1..9: note = Complementarity-determining region (CDR)
QHYYDYPRT

SEQ ID NO: 31 - protein, 8 aa, synthetic construct
  REGION 1..8: note = Complementarity-determining region (CDR)
GFSLTNYA

SEQ ID NO: 32 - protein, 6 aa, synthetic construct
  REGION 1..6: note = Complementarity-determining region (CDR)
WTGGGT

SEQ ID NO: 33 - protein, 20 aa, synthetic construct
  REGION 1..20: note = Complementarity-determining region (CDR)
ARVSHDYFYGSSPYYYGMDY

SEQ ID NO: 34 - protein, 6 aa, synthetic construct
  REGION 1..6: note = Complementarity-determining region (CDR)
QNINVW

SEQ ID NO: 35 - skipped

SEQ ID NO: 36 - protein, 9 aa, synthetic construct
  REGION 1..9: note = Complementarity-determining region (CDR)
QQGQSYPWT

SEQ ID NO: 37 - protein, 488 aa, synthetic construct
  REGION 1..488: note = n418 vh amino acid sequence
MGWSCIILFLVATATGEVQLVEKGGGLVQPGKSLKLSCAASGFTFSEYWMNWVRQAPGKGLEWVGVIKYKYSNYATEYAESVKGRFTISRDDSKSSVYLQMNNLRAEDTAIYYCARTWENWYFDFWGQGTQVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDRSEPKSCDKTHTCPPCPAPELLGDDSVFLFPPKPKDTLMISRTPEVTCVVVDVSDEDGEVKFNWYVDGVEVHNAKTKPREEQYNSTYRVVSVLTVLHQDWLNGKEYKCKVSNKALPRPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKSAWSHPQFEK

SEQ ID NO: 38 - protein, 107 aa, synthetic construct
  REGION 1..107: note = n418 vl amino acid sequence
DIQMTQSPSSLPASLGDRVTIHCQASQDISNYLTWYQQKPGKAPKLLIYETNKLADGVPSRFSGSGSGRDYSFTISSLESEDVGSYYCQHYYDYPRTFGPGTKLEIK

SEQ ID NO: 39 - protein, 126 aa, synthetic construct
  REGION 1..126: note = hd109 vh amino acid sequence
QVQLTESGPGLVAPSQSLSITCSVSGFSLTNYAINWVRQPPGKGLEWLGIIWTGGGTNYNSTLKSRLSISKDNSKSQVFLKMNSLQTDDTARYYCARVSHDYFYGSSPYYYGMDYWGQGTSVTVSS

SEQ ID NO: 40 - protein, 107 aa, synthetic construct
  REGION 1..107: note = hd109 vl amino acid sequence
DIQMNQSPSSLSASLGDTITITCHASQNINVWLSWYQQKPGNIPKLLIYKVSNLHTGVPSRFSGSGSGTGFTLTISSLQPEDIATYYCQQGQSYPWTFGGGTKLEIK

SEQ ID NO: 41 - protein, 277 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 42 - protein, 261 aa, Homo sapiens
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 43 - DNA, 19 bp, synthetic construct
  misc_feature 1..19: note = Single strand DNA oligonucleotide
  source 1..19: mol_type = other DNA
tcatttaccaggagagtgg

SEQ ID NO: 44 - DNA, 29 bp, synthetic construct
  misc_feature 1..29: note = Single strand DNA oligonucleotide
  source 1..29: mol_type = other DNA
agaggctcttctcagtatggtggttgtgc

SEQ ID NO: 45 - DNA, 24 bp, synthetic construct
  misc_feature 1..24: note = Single strand DNA oligonucleotide
  source 1..24: mol_type = other DNA
gctcacgtccaccaccacacatgt

SEQ ID NO: 46 - DNA, 24 bp, synthetic construct
  misc_feature 1..24: note = Single strand DNA oligonucleotide
  source 1..24: mol_type = other DNA
gaaatagcccttgaccaggcatcc

SEQ ID NO: 47 - DNA, 20 bp, synthetic construct
  misc_feature 1..20: note = Single strand DNA oligonucleotide
  source 1..20: mol_type = other DNA
aacactcattcctgttgaag

SEQ ID NO: 48 - DNA, 28 bp, synthetic construct
  misc_feature 1..28: note = Single strand DNA oligonucleotide
  source 1..28: mol_type = other DNA
gctcttgacaatgggtgaagttgatgtc

SEQ ID NO: 49 - DNA, 28 bp, synthetic construct
  misc_feature 1..28: note = Single strand DNA oligonucleotide
  source 1..28: mol_type = other DNA
ctaacactcattcctgttcagggtcttg

SEQ ID NO: 50 - DNA, 24 bp, synthetic construct
  misc_feature 1..24: note = Single strand DNA oligonucleotide
  source 1..24: mol_type = other DNA
gctgctcaggctgtaggtgctgtc

SEQ ID NO: 51 - protein, 8 aa, synthetic construct
  REGION 1..8: note = short synthetic peptide
SIINFEKL

SEQ ID NO: 52 - protein, 8 aa, synthetic construct
  REGION 1..8: note = Complementarity-determining region (CDR)
GFTFSNAA

SEQ ID NO: 53 - protein, 10 aa, synthetic construct
  REGION 1..10: note = Complementarity-determining region (CDR)
IRTRPSKYAT

SEQ ID NO: 54 - protein, 11 aa, synthetic construct
  REGION 1..11: note = Complementarity-determining region (CDR)
TPRATEDVPFY

SEQ ID NO: 55 - protein, 12 aa, synthetic construct
  REGION 1..12: note = Complementarity-determining region (CDR)
QSVLYDENKKNY

SEQ ID NO: 56 - skipped

SEQ ID NO: 57 - protein, 9 aa, synthetic construct
  REGION 1..9: note = Complementarity-determining region (CDR)
QQYYDFPPT

SEQ ID NO: 58 - protein, 8 aa, synthetic construct
  REGION 1..8: note = Complementarity-determining region (CDR)
GYTFTNYW

SEQ ID NO: 59 - skipped

SEQ ID NO: 60 - protein, 9 aa, synthetic construct
  REGION 1..9: note = Complementarity-determining region (CDR)
QQYYDFPPT

SEQ ID NO: 61 - protein, 11 aa, synthetic construct
  REGION 1..11: note = Complementarity-determining region (CDR)
QSLVHSNGNTY

SEQ ID NO: 62 - skipped

SEQ ID NO: 63 - protein, 9 aa, synthetic construct
  REGION 1..9: note = Complementarity-determining region (CDR)
SQNTHVPYT